(19) United States
(12) Patent Application Publication
(10) Pub. No.: US 2025/0011790 A1
(43) Pub. Date: Jan. 9, 2025

(54) COMBINED TARGETING OF STAT3 AND ULKI TO TREAT GLIOBLASTOMA

(71) Applicants: Vanderbilt University, Nashville, TN (US); University of Tennessee Research Foundation, Knoxville, TN (US)

(72) Inventors: Edward CHAUM, Nashville, TN (US); Sujoy BHATTACHARYA, Nashville, TN (US); Lawrence PFEFFER, Nashville, TN (US)

(21) Appl. No.: 18/709,175

(22) PCT Filed: Nov. 11, 2022
(86) PCT No.: PCT/US2022/079700

§ 371 Date: May 10, 2024

Related U.S. Application Data
(60) Provisional application No. 63/278,317, filed on Nov. 11, 2021.

(51) Int. Cl. C12N 15/113

(52) U.S. Cl. CPC C12N 15/1137; C12N 2310/11

(57) ABSTRACT

Disclosed herein is a composition comprising a signal transducer and activator of transcription 3 (STAT3) inhibitor and one or more of a UNC51-like 1 (ULK1) inhibitor, histone deacetylase (HDAC) inhibitor, or an mTOR inhibitor in a pharmaceutically acceptable carrier. Also disclosed is a method for treating glioblastoma in a subject that involves administering to the subject an effective amount of a STAT3 inhibitor and one or more of a ULK inhibitor, an AMPKα inhibitor, an HDAC inhibitor, or an mTOR inhibitor.

### CROSS-REFERENCE TO RELATED APPLICATIONS

This application claims benefit of U.S. Provisional Application No. 63/278,317, filed Nov. 11, 2021, which is hereby incorporated herein by reference in its entirety.

### BACKGROUND

Despite advanced molecular characterization, glioblastoma multiforme (GBM) remains the most lethal type of brain tumor with high mortality rates in both pediatric and adult patients. The high tumor-initiating capabilities of the glioblastoma cancer stem cells (GSCs) and their ability to evade therapy contribute to GBM maintenance and tumor relapse.

### SUMMARY

The transcription factor, STAT3, is a well-recognized oncogenic driver of glioblastoma, and STAT3 has been recently implicated in autophagy, a central cellular process linked to GSC aggressiveness. The role of autophagy in cancer is controversial because it has been reported to both promote and inhibit tumorigenesis. However, autophagy is emerging as a crucial player in GBM pathobiology. Autophagy suppression has been associated with the maintenance of stemness, invasiveness, and plasticity of GSCs, but it is unknown whether manipulation of autophagy will reduce GBM tumorigenesis and tumor relapse. Therefore, identifying autophagy pathways critical for GSC pathogenesis is of utmost importance in achieving long-lasting antitumor effects and improving patient survival.

Disclosed herein is a composition comprising a signal transducer and activator of transcription 3 (STAT3) inhibitor and one or more of a UNC51-like 1 (ULK1) inhibitor, histone deacetylase (HDAC) inhibitor, or an mTOR inhibitor in a pharmaceutically acceptable carrier.

For example, in some embodiments, the STAT3 inhibitor is WP1066. In some embodiments, the one or more of a UNC51-like 1 (ULK1) inhibitor, histone deacetylase (HDAC) inhibitor, or an mTOR inhibitor allows for a reduction in the dosage of the STAT3 inhibitor. For example, the STAT3 inhibitor WP1066 has been used in clinical trials at 8-16 mg/kg, twice a day. As disclosed herein, the combination of WP1066 and a UL1 inhibitor is effective at a WP1066 dosage of 4 mg/kg body weight.

In some embodiments, the ULK1 inhibitor is MRT68921 or SB10206965. In some embodiments, the AMPKα inhibitor is Dorsomorphin. In some embodiments, the HDAC inhibitor is an HDAC 1, 2, 3, and 6 inhibitor. In some embodiments, the HDAC inhibitor comprises Vorinostat (SAHA). In some embodiments, the HDAC inhibitor comprises Trichostatin A. In some embodiments, the mTOR inhibitor comprises rapamycin or a rapalog. In some embodiments, the mTOR inhibitor comprises Everolimus. In some embodiments, the mTOR inhibitor comprises RapaLink.

Also disclosed is a method for treating glioblastoma in a subject that involves administering to the subject an effective amount of a STAT3 inhibitor and one or more of a ULK inhibitor, an AMPKα inhibitor, an HDAC inhibitor, or an mTOR inhibitor.

In some embodiments, the STAT3 inhibitor and the ULK1 inhibitor, AMPKα inhibitor, HDAC inhibitor, or mTOR inhibitor are administered concurrently. In some embodiments, the ULK1 inhibitor, AMPKα inhibitor, HDAC inhibitor, or mTOR inhibitor are administered in the same composition.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1H show STAT3 deletion activates autophagy through AMPKα-ULK1-TSC2 signaling pathways in MT330 cells. FIG. 1A shows confluent EV MT330 cells, STAT3-knockout cell line #2 (KO2), STAT3-knockout cell line #3 (KO3), STAT3-KO3 rescued with wild-type (WT), and STAT3-KO3 cells expressing Y705F-STAT3 and S727A-STAT3 mutants were exposed to Bafilomycin (Baf, 100 nM) for 3 h. Untreated (UT) cells served as controls. Total cell lysates were prepared and immunoblotted with indicated antibodies. FIG. 1B shows quantification of the ratio of phospho-STAT3, and total-STAT3 from three independent experiments. FIG. 1C shows cell lysates analyzed for p-AMPKα T172. Blots were stripped and probed for total-AMPKα. FIG. 1D shows quantification of the ratio of phosphorylated and total AMPKα shown in FIG. 1C. FIG. 1E shows cell lysates analyzed for p-ULK1 S555 and S638. Blots were stripped and probed for total-ULK1. FIG. 1F shows quantification of the ratio of phospho-ULK1 S555 and total-ULK1. FIG. 1G shows Western blotting of cell lysates with phospho-TSC2 antibodies. Blots were stripped and probed with total-TSC2 antibody. FIG. 1H shows quantification of the ratio of phospho-T1462- and total-TSC2 and phospho-S1387 and total-TSC2.

FIGS. 2A to 2I show STAT3 Represses autophagy in LN229 cells. FIG. 2A shows EV LN229 cells, STAT3-knockout (KO), STAT3-KO cells rescued with WT STAT3, and STAT3-KO cells expressing Y705F-STAT3 and S727A-STAT3 phosphorylation-defective mutants, were exposed to Bafilomycin (Baf, 100 nM) for 3 h or left untreated (UT). Total cell lysates were prepared and immunoblotted with indicated antibodies with p-Actin serving as a loading control. FIG. 2B shows densitometric analysis from n=3 observations of the ratio of phosphorylated STAT3 to total-STAT3 shown in FIG. 2A.

FIG. 2C shows EV, STAT3-KO and STAT3 mutant expressing lines with treated with or without 100 nM bafilomycin for 3 h. Cell lysates were immunoblotted for LC3-I/II with Actin as a loading control. FIG. 2D shows quantification of data shown in FIG. 2C. FIG. 2E shows cell lysates were analyzed for p-AMPKα Thr172. Blots were stripped and probed for total-AMPKα. FIG. 2F shows quantification of the ratio of phosphorylated and total AMPKα shown in FIG. 2E. FIG. 2G shows cell lysates immunoblotted with the indicated antibodies. FIG. 2H shows cell lysates were analyzed for phospho-ULK1 S555 and total-ULK. FIG. 2I shows quantification of data shown in FIG. 2H.

FIGS. 3A to 3H show STAT3-KO activates autophagy through mTOR-independent but Prom1-dependent signalling pathways in MT330 cells. FIG. 3A shows EV MT330 cells, STAT3-KO, STAT3-KO rescued with WT-STAT3, and STAT3-KO cells expressing Y705F (Y) and S727A (S) STAT3 mutants were treated with or without 100 nM bafilomycin for 3 h. Cell lysates were analysed for LC3-I/II, p62 and CathepsinD.

FIGS. 3B and 3C show quantification of LC3-II/Actin ratio (FIG. 3B) and p62/Actin ratio (FIG. 3C). FIG. 3D shows DMSO, Everolimus (RAD001) (10 μM) and RAD001 (10 μM)+Baf (100 nM) for 3 h. Cell lysates were immunoblotted with the indicated antibodies and beta actin was used as an internal loading control. FIG. 3E shows quantification of phospho-mTOR S2448 and total-mTOR ratio. FIG. 3F shows quantification of phospho-S6 Ribosomal protein (Rbp) S235/236 and total-S6 Rbp ratio. FIG. 3G shows quantification of LC3-II/Actin ratio presented in FIG. 3D. FIG. 3H shows EV, STAT3-KO cells, KO3 rescued with WT-STAT3, and KO3 cells expressing Y705F and S727A mutants grown to confluence and treated with Baf (100 nM) for 3 h. Cell lysates were analyzed by Western blotting using antibodies specific for STAT3, Prom1 and LC3-I/II.

FIGS. 4A to 4J show transcriptional regulation of autophagy-associated genes. FIG. 4A shows EV MT330 cells, STAT3-KO and STAT3-KO cells rescued with WT-STAT3, or phosphorylation-defective mutants were analyzed for BNIP3 expression by Western blotting. Blot was stripped and probed for actin. FIG. 4B shows quantification of data shown in FIG. 4A. FIG. 4C shows fold change in BNIP3 gene expression in MT330 cells. FIG. 4D shows MT330 cells were analysed for p62 and ULK1 protein levels. Blots were stripped and probed for actin. FIGS. 4E and 4F show fold change in p62 (FIG. 4E) and ULK1 (FIG. 4F) genes in MT330 cells. FIG. 4G shows analysis of p62 protein levels in LN229 cells. Blot was stripped and probed for actin. FIG. 4H shows quantification of data shown in FIG. 4G. FIGS. 4I and 4J show fold change in p62 (FIG. 4I) and ULK1 (FIG. 4J) genes in LN229 cells,

FIG. 5 shows immunolocalization of LC3 and p62 in MT330 cells. EV MT330 cells, STAT3-KO, STAT3-KO cells rescued with WT-STAT3, were grown on chamber slides, and treated with or without 1 μM Baf for 48 h. Cells were fixed and immunostained for LC3, p62, and counterstained with DAPI, and analysed by confocal microscopy. Scale bar 20 μM.

FIGS. 6A to 6F show pharmacologic inhibition of ULK1 activity inhibits autophagy and induces apoptosis in STAT3-KO MT330 cells. FIG. 6A shows MT330 EV, STAT3-KO, WT, Y705F-STAT3 (Y), and S727A-STAT3 (S) mutant expressing cells treated with the ULK1 inhibitor, MRT68921 (20 μM), in the presence or absence of Baf (100 nM) for 5 h. Total cell lysates were immunoblotted with indicated antibodies with p-Actin serving as a loading control. FIGS. 6B to 6E show quantification of the ratio of phospho- and total-ATG14 (FIG. 6B); phospho- and total-Beclin1 (FIG. 6C); phospho- and total-ULK1 (FIG. 6D); and LC3-II and Actin (FIG. 6E). FIG. 6F shows apoptotic cell death (DNA fragmentation) measured by ELISA after treatment of MT330 cells with 20 μM MRT68921 for 5 h (mean SE, n=3).

FIGS. 7A to 7E show knockdown of ULK1 expression blocks autophagy and induces apoptosis in STAT3-KO and STAT3-mutant expressing lines. FIG. 7A shows MT330 EV, STAT3-KO, WT, Y705F and S727A mutant expressing cells grown to confluence and treated with control or ULK1 siRNA followed by treatment with or without Baf for 3 h. ULK1 knockdown was verified by Western blotting with ULK1 and phospho-ULK1, and cell lysates were analysed for LC3, cleaved caspase-3 with p-Actin as a loading control. FIGS. 7B to 7D show quantification of the ratio of total-ULK1 and Actin (FIG. 7B); LC3-II and Actin (FIG. 7C); active caspase-3 and Actin (FIG. 7D). FIG. 7E shows apoptosis was measured by ELISA as in FIG. 6F (mean SE, n=3).

FIG. 8 is a schematic representation of the molecular cross talk in STAT3-dependent autophagy and chemoresistance in GBM. →, Activation; ⊥, inhibition; sahded (P) indicates phosphorylation causing inhibition; and open (P) indicates phosphorylation leading to activation. Boxes in green indicate protein activation, and boxes in red indicate inhibition.

FIGS. 9A to 9C show post-translational modifications (acetylation and tri-methylation) of STAT3 in GBM cells. FIG. 9A shows control EV MT330 and LN229 cells, STAT3-KO, and KO-cells reconstituted with WT and phosphoryation-defective Y705F-STAT3 and S727A-STAT3 mutants were exposed to bafilomycin (Baf, 100 nM) for 3 h. Cell lystaes were immunoblotted with Acetyl (Ac)-STAT3 K685 antibody. FIG. 9B shows cells lysates immunoprecipitated (IP) with STAT3 and immunoprecipitates were western blotted (WB) with Tri-methyl-K180 STAT3. Membranes were stripped and probed for total-STAT3. FIG. 9C shows quantification of Trimethyl- to total-STAT3.

FIG. 10 shows Kaplan-Meier analysis of survival data in mice showing EV MT330 triggers GBM tumors and decreases survival in mice. STAT3-KO and Y705F-STAT3 mutant expressing MT330 cells increase survival in mice.

FIG. 11 shows CD133/Prom1 gene expression in GBM6 and X16 Glioblastoma Cancer Stem Cells.

FIG. 12 shows CD133/Prom1 cell surface expression in GBM6 and X16 Glioblastoma Cancer Stem Cells.

FIG. 13 shows CD133/Prom1 and CD44 cell surface expression in GBM6 and X16 Glioblastoma Cancer Stem Cells.

FIG. 14 shows CD133-ve glioblastoma stem cells (GSCs) have low pY705-STAT3, and elevated autophagy compared to CD133+ve GSCs suggesting that cell surface CD133 is negatively associated with autophagy in GSCs.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof. In some embodiments, an active agent is a nucleic acid, e.g., miRNA or a derivative or variant thereof.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Disclosed herein are compositions and methods for treating glioblastoma using a combination of STAT3 inhibitors and ULK1 inhibitors.

STAT3 Inhibitors

The most common approach in targeting STAT3 directly is to prevent the formation of functional STAT3 dimers through disrupting the domains of SH2, DBD, or NTD. In general, direct inhibitors of STAT3 can be classified into three categories: peptides, small molecules and oligonucleotides. Studies of these inhibitors on pre-clinical cancer models and clinical trials are summarized in Table 1.

TABLE 1
STAT3 inhibitors

| | | |
|---|---|---|
| Direct | Peptides | DBD-1 |
| Inhibitors | | ISS-610 prodrugs |
| | | PY*LKTK |
| | Small molecules | BBI608 (FDA approved) |
| | | Celecoxib* (FDA |
| | | approved) |
| | | C188-9 |
| | | OPB-111077 |
| | | OPB-31121 |
| | | OPB-51602 |
| | | Pyrimethamine* (FDA |
| | | approved) |
| | | 6o |
| | | FLLL32 |
| | | HJC0152 |
| | | LL1 |
| | | LLL-3 |
| | | LLL12 |
| | | LYW-6 |
| | | Nitidine chloride |
| | | SD-36 |
| | | Stattic |
| | | STX-0119 |
| | | S3I-1757 |
| | | S3I-201 |
| | | CPA-7 |
| | | C48 |
| | | GPA512 |
| | | MMPP |
| | Oligonucleotides | AZD9150 |
| | | STAT3 decoy |
| | | InS3-54A18 |
| | | STAT3 hpdODN |
| Indirect | JAK2 | INCB16562 |
| Inhibitors | | TG101209 |
| | | LY2784544 |
| | | SB1518 |
| | JAK1/2 | AZD-1480 |
| | | CYT 387 |
| | | Ruxolitinib (FDA approved) |
| | EGFR | Cetuximab (FDA |
| | | approved) |
| | | Panitumumab (FDA |
| | | approved) |
| | | JND3229 |
| | FGFR | Ponatinib (FDA approved) |
| | IL-6R | Siltuximab (FDA approved) |
| | | Tocilizumab (FDA |
| | | approved) |
| | VEGF | Bevacizumab (FDA |
| | | approved) |
| | VEGFR | Apatinib |
| | VEGFR, PDGFR | Sorafenib (FDA approved) |
| | VEGFR, PDGFR, c-KIT | Sunitinib (FDA approved) |
| | FGFR, VEGFR | ODM-203 |
| | SRC, ABL | Dasatinib (FDA approved) |
| | SRC | Bosutinib (FDA approved) |
| | | KX2-391 |

Peptides are usually designed based on the structure of amino acid residues in STAT3 protein and can be directed towards different domains. Phosphopeptide inhibitor (PY*LKTK), derived from the binding peptide sequence of the STAT3-SH2 domain, represents the first successful attempt to disrupt STAT3 dimerization.

Non-peptide small molecules capable of disrupting phosphorylation of STAT3 or STAT3-STAT3 dimerization have recently emerged as an attractive alternative approach to the above. These small molecule inhibitors usually selectively bind to the SH2, the DBD, or the NTD domain of STAT3 to block transcription of target genes. Numerous small molecule inhibitors of STAT3 have also been identified by virtual screening and proteolysis-targeting chimera (PROTAC)-based strategies.

STAT3-binding decoy oligodeoxynucleotides can sequester STAT3 and thus decrease its binding to cognate DNA sites within target genes. Antisense oligonucleotides (ASOs) are designed to block STAT3 activity by targeting STAT3 mRNA. For example, AZD9150, a second-generation STAT3 ASO, targets the 3′-untranslated region (3′-UTR) of the STAT3 gene. Preclinical testing and clinical evaluation have revealed the high efficacy and low toxicity of AZD9150 in oncotherapy. Aptamers have also emerged as useful targeted delivery agents for conventional drugs and small RNAs including siRNAs and miRNAs due to several advantages, such as small physical size, high stability and low immunogenicity. Recently, STAT3 silencing by aptamer-siRNA chimera obtained excellent inhibition in the therapy of glioblastoma, suggesting that the improved oligonucleotides might offer translational potential for the treatment of solid tumors.

Some FDA-approved compounds, such as Pyrimethamine and Celecoxib, have been identified as STAT3 inhibitors through drug-repositioning screening. These findings not only provide another source for searching STAT3 inhibitors, but also suggest potential applications of these drugs in cancer therapy. In addition, similar to combined therapy, certain bifunctional compounds are emerging and may represent a new generation of highly efficacious STAT3 inhibitors for cancer therapy in the future. For example, the compound 8u has dual immunotherapeutic and anticancer efficacy through simultaneously inhibiting indoleamine-2,3-dioxygenase 1 (IDO1) and STAT3.

In parallel with direct inhibitors, indirect inhibitors of STAT3 have been pursued by targeting the upstream or downstream components of the STAT3 signaling pathway, and hundreds of leading compounds have been identified. Out of those, Ruxolitinib, Dasatinib and Siltuximab that target JAK, SRC/ABL, and IL-6 respectively, have been approved by FDA for cancer therapy. Indirect STAT3 inhibitors in currently on-going clinical trials are summarized in Table 1.

Intriguingly, it has recently been shown that phosphorylated STAT3 is present in exosomes from 5-fluorouracil (5-FU) resistant colorectal cancer cells, which contributes to acquired 5-FU resistance. Given the importance and efficiency of exosomes in intercellular and interorgan communication, these findings not only add another complexity to STAT3 regulation, but also pave a new way to inhibit the oncogenic function of STAT3, as well as to delivery STAT3 inhibitors via exosomes.

STAT3 modulators are also described in US2011/0144043, which is incorporated by references for these inhibitors and methods of making and using same. For example, in some embodiments, the STAT3 inhibitor is selected from the group consisting of pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, and 3-(2-hydroxyphenyl)-3-phenyl-N,N-dipropylpropanamide as well as any derivatives of these compounds or analogues thereof. These compounds are commercially available through various sources.

STAT3 inhibitors are further described in U.S. Pat. No. 9,884,863, which is incorporated by references for these inhibitors and methods of making and using same. For example, in some embodiments, the STAT3 inhibitor is a compound having the structure:

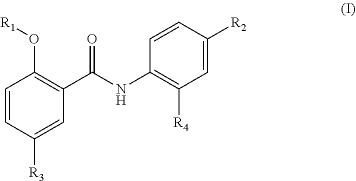

wherein R1 is selected from the group consisting of hydrogen, substituted or unsubstituted alkyl(C1-12), substituted or unsubstituted heterocycloalkyl(C4-12), acyl(C1-C6), alkylamino(C1-6), alkoxyamino(C1-6),

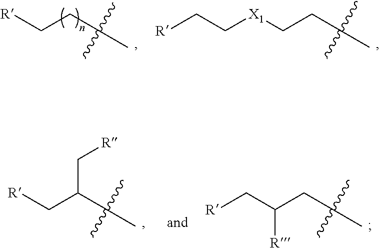

wherein X1 is —O— or —NH—; wherein n=0, 1, 2, 3, 4, or 5; wherein R′ is selected from the group consisting of substituted or unsubstituted alkyl(C1-6), alkylamino(C1-6), halogen, —OH, amido(C1-12), alkyl sulfonylamino(C1-12),

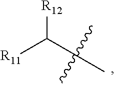

substituted or unsubstituted heterocycloalkyl(C4-12); wherein R11 and R12 are each independently —H or alkyl(C1-6); wherein R″ is —H, —OH, —NH2, or halogen; wherein R′″ is alkyl(C1-6);
wherein if R2 is —NO2, and R3 and R4 are —Cl, then R1 is not —H; wherein R2 is selected from the group consisting of —NO2, —NH2, H, amido(C1-12), substituted amido(C1-12), alkylsulfonylamino(C1-12), dialkylsulfonylamino(C1-12), and halogen; wherein R3 and R4 are halogen.

For example, in some embodiments, the STAT3 inhibitor is a compound having the structure:

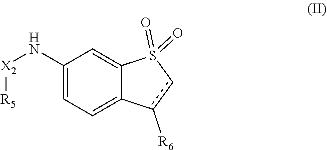

wherein R5 is selected from the group consisting of alkyl(C1-14), substituted alkyl(C1-14), —H,

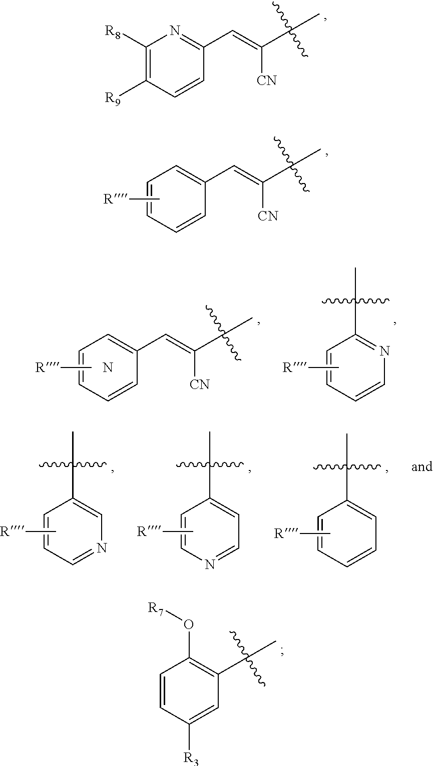

wherein R3 is halogen, —OH, or —H; wherein R7 is —H or R1; wherein R8 and R9 are each independently —H or halogen; wherein R6 is —H or —O—CH3; wherein X2 is —C(O)— or —S(O)2-; wherein R′″ is —H, —CF3, —NO2, —CN, halogen, alkyl(C1-12), alkoxy(C1-12), aryl(C6-12), heteroaryl(C4-12), substituted carboxylates(C1-C12), amido(C1-C12), substitutedalkylamino(C1-C12), or —S(O)2-X3; wherein X3 is —OH, —NH2, —OCH3, —OCH2CH3, —C(O)CH3, —NHCH3, —NHCH2CH3, —N(CH3)2, —C(O)NH2, or —OC(O)CH3.

For example, in some embodiments, the STAT3 inhibitor is a compound having the structure:

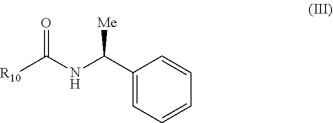

wherein R10 is substituted or unsubstituted aryl(C6-18), substituted or unsubstituted heteroaryl(C6-18), or R5.

For example, in some embodiments, the STAT3 inhibitor is a compound having the structure:

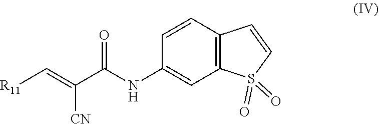

wherein R11 is substituted or unsubstituted aryl(C6-18), substituted or unsubstituted heteroaryl(C6-18), cycloalkyl(C6-12), or R5; or a salt thereof.

### ULK Inhibitors

The ULK (UNC51-like) enzymes are a family of mammalian kinases that have critical roles in autophagy and development. The ULK family of kinases comprises 5 genes in mammals: ULK1 through ULK4 and STK36. In mammals, ULK1 and ULK2 have been shown to be necessary for the proper autophagy induction and contribute to various developmental, physiological, and pathological processes.

The serine/threonine-protein kinases ULK1 and ULK2 are evolutionarily conserved serine/threonine kinase orthologs of the yeast autophagy related (Atg) family member Atg1, that have redundant roles in the regulation of autophagy. Autophagy targets long-lived proteins or organelles for degradation in lysosomes, and the products of this process are then recycled for other cellular pathways. The canonical ULK/Atg1 complex is composed of ULK1, ATG13, RB1CC1/FIP200/ATG17, and ATG101. It initiates autophagosome formation, at least in part by phosphorylating components of the autophagy-inducing class Ill phosphatidylinositol 3-kinase complex (e.g., PI3K3C/Vps34, PIK3R4/Vps15, BECN1/Vps30/ATG6, ATG14). ULK/Atg1 also promotes membrane recycling via ATG9. Consistent with the established role of ULK1/2 in autophagy, disrupting ULK1 expression in mice results in a defect in autophagy-mediated clearance of mitochondria during red blood cell maturation, and mice lacking both ULK1 and ULK2 expression die shortly after birth due to a defect in glycogen metabolism, which is similar to other autophagy-defective mice.

SBI-0206965 is a potent, selective and cell permeable autophagy kinase ULK1 inhibitor with IC₅₀s of 108 nM for ULK1 kinase and 711 nM for the highly related kinase ULK2, having the structure:

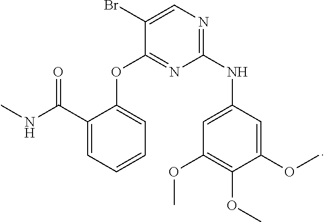

MRT67307 is a dual inhibitor of the IKKE and TBK-1, having the structure:

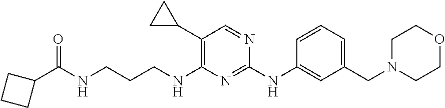 [SMILES: O=C(NCCCNc1nc(Nc2cccc(CN3CCOCC3)c2)ncc1C1CC1)C1CCC1]

MRT67307 also inhibits ULK1 and ULK2 with IC₅₀s of 45 and 38 nM, respectively.

MRT68921 dihydrochloride is a potent inhibitor of ULK1 and ULK2, with IC₅₀ values of 2.9 nM and 1.1 nM, respectively, having the structure:

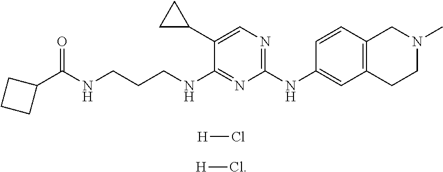

ULK-101 is a potent and selective ULK1 inhibitor, with IC50 values of 1.6 nM and 30 nM for ULK1 and ULK2, respectively, having the structure:

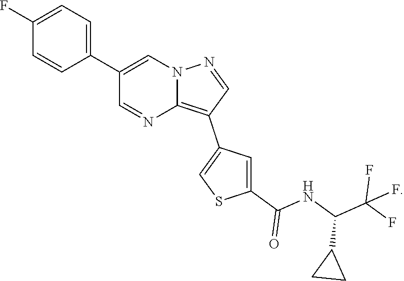 [SMILES: O=C(N[C@@H](C1CC1)C(F)(F)F)c1cc(-c2cnn3cc(-c4ccc(F)cc4)cnc23)cs1]

XST-14 is a potent, competitive and highly selective ULK1 inhibitor with an IC₅₀ of 26.6 nM having the structure:

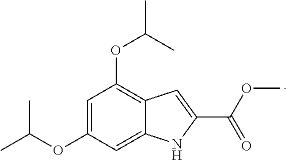 [SMILES: COC(=O)c1cc2c(OC(C)C)cc(OC(C)C)cc2[nH]1]

XST-14 induces autophagy inhibition by reducing the phosphorylation of the ULK1 downstream substrate. XST-14 induces apoptosis in hepatocellular carcinoma (HCC) cells and has antitumor effects.

SBP-7455 is a potent, high affinity and orally active dual ULK1/ULK2 autophagy inhibitor with IC₅₀s of 13 nM and 476 nM in the ADP-Glo assays, respectively, having the structure:

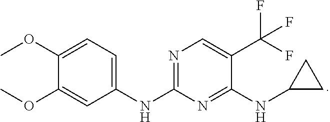 [SMILES: COc1ccc(Nc2ncc(C(F)(F)F)c(NC3CC3)n2)cc1OC]

GW406108X is a specific Kif15 (Kinesin-12) inhibitor with an IC₅₀ of 0.82 uM in ATPase assays, having the structure:

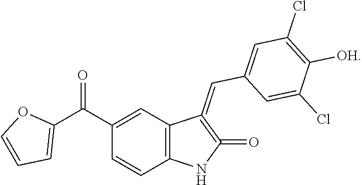

GW406108X, a potent autophagy inhibitor, shows ATP competitive inhibition against ULK1 with a pIC₅₀ of 6.37 (427 nM). GW406108X inhibits ULK1 kinase activity and blocks autophagic flux, without affecting the upstream signaling kinases mTORC1 and AMPK.

MRT68921 is a potent inhibitor of ULK1 and ULK2, with IC₅₀ values of 2.9 nM and 1.1 nM, respectively, having the structure:

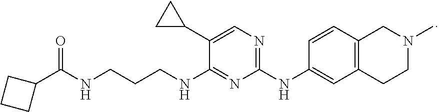 [SMILES: CN1CCc2cc(Nc3ncc(C4CC4)c(NCCCNC(=O)C4CCC4)n3)ccc2C1]

MRT67307 hydrochloride is a dual inhibitor of the IKKε and TBK-1 with IC₅₀s of 160 and 19 nM, respectively, having the structure:

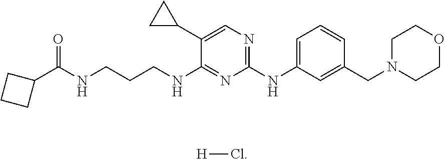 [SMILES: O=C(NCCCNc1nc(Nc2cccc(CN3CCOCC3)c2)ncc1C1CC1)C1CCC1 ; Cl]

MRT67307 hydrochloride also inhibits ULK1 and ULK2 with IC₅₀s of 45 and 38 nM, respectively. MRT67307 hydrochloride also blocks autophagy in cells.

### AMPKα Inhibitor

AMP-activated protein kinase (AMPK) is an evolutionarily conserved energy sensor important for cell growth, proliferation, survival, and metabolic regulation. Active AMPK inhibits biosynthetic enzymes like mTOR and acetyl CoA carboxylase (required for protein and lipid synthesis, respectively) to ensure that cells maintain essential nutrients and energy during metabolic crisis. Despite our knowledge about this incredibly important kinase, no specific chemical inhibitors are available to examine its function. However, one small molecule known as compound C (also called dorsomorphin) has been widely used in cell-based, biochemical, and in vivo assays as a selective AMPK inhibitor. In nearly all these reports including a recent study in glioma, the biochemical and cellular effects of compound C have been attributed to its inhibitory action toward AMPK.

Therefore, in some, the AMPK inhibitor is do (dorsomorphin) (compound C), having the structure:

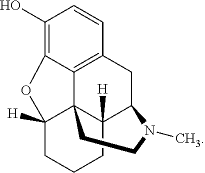

In some, the AMPK inhibitor is (S)-4-(2-(4-Amino-1,2,5-oxadiazol-3-yl)-1-ethyl-7-(piperidin-3-ylmethoxy)-1H-imidazo[4,5-c]pyridin-4-yl-2-methylbut-3-yn-2-ol (GSK 690693), having the structure:

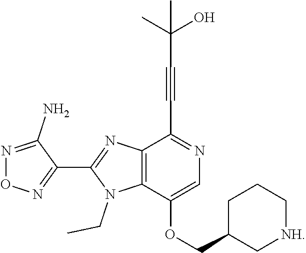 [SMILES: CCn1c(-c2nonc2N)nc2c(C#CC(C)(C)O)ncc(OC[C@H]3CCCNC3)c21]

GSK690693 is a pan-Akt inhibitor targeting Akt1/2/3 with IC50 of 2 nM/13 nM/9 nM in cell-free assays, also sensitive to the AGC kinase family: PKA, PrkX and PKC isozymes. GSK690693 also potently inhibits AMPK and DAPK3 from the CAMK family with IC₅₀ of 50 nM and 81 nM, respectively.

### HDAC Inhibitor

Some exemplary inhibitors of HDAC include small molecular weight carboxylates (e.g., less than about 250 amu), hydroxamic acids, benzamides, epoxyketones, cyclic peptides, and hybrid molecules. (See, for example, Drummond D. C., et al. Annu. Rev. Pharmacol. Toxicol. (2005) 45: 495-528, (including specific examples therein) which is hereby incorporated by reference in its entirety). Non-limiting examples HDAC inhibitors include, but are not limited to, Suberoylanilide Hydroxamic Acid (SAHA (e.g., MKO683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., Cl-994 (i.e., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (i.e., 6-(3-chlorophenylureido)caproic hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. HDAC inhibitors are commercially available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich. Further HDAC inhibitors amenable to the invention include, but are not limited to, those that are described in U.S. Pat. Nos. 7,183,298; 6,512,123; 6,541,661; 6,531472; 6,960,685; 6,897,220; 6,905,669; 6,888,207; 6,800,638 and 7,169,801, and U.S. patent application Ser. Nos. 10/811,332; 12/286,769; 11/365,268; 11/581,570; 10/509,732; 10/546,153; 10/381,791 and 11/516,620, the contents of which each are incorporated herein by reference in their entirety.

mTOR Inhibitor

Rapamycin and rapalogs (rapamycin derivatives) are small molecule inhibitors, which have been evaluated as anticancer agents. The rapalogs have more favorable pharmacokinetic profile compared to rapamycin, the parent drug, despite the same binding sites for mTOR and FKBP12. Rapamycin is the main mTOR inhibitor, but deforolimus (AP23573), everolimus (RAD001), and temsirolimus (CCI-779), are the newly developed rapamycin analogs.

In some embodiments, the rapalog is sirolimus, having the structure:

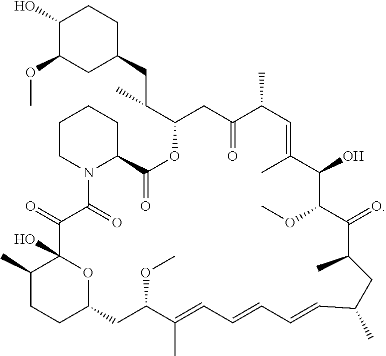 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the rapalog is temsirolimus (CCI-779), having the structure:

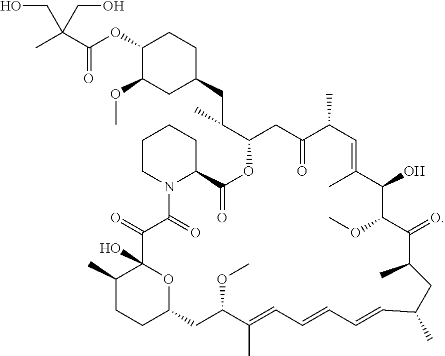 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

Temsirolimus is a prodrug of rapamycin. It is approved by the U.S. Food and Drug Administration (FDA) and the European Medicines Agency (EMA), for the treatment of renal cell carcinoma (RCC).

In some embodiments, the rapalog is everolimus (RAD001), having the structure:

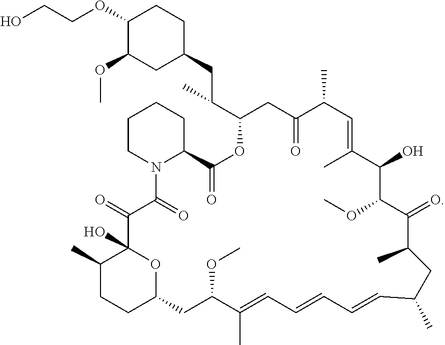 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the rapalog is Ridaforolimus (AP23573, MK-8669), having the structure:

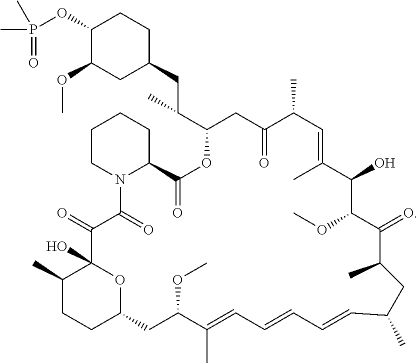 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](O)[C@@H](OC)C(=O)[C@H](C)C[C@H](C)/C=C/C=C/C=C/1C]

### Pharmaceutical Compositions and Methods

Pharmaceutical compositions disclosed herein comprise an effective amount of an agent disclosed herein (e.g. STAT3 inhibitor and/or a ULK inhibitor) in a pharmaceutically acceptable carrier. In some embodiments, a STAT3 inhibitor and/or a ULK inhibitor may be conjugated with a pharmaceutically acceptable carrier such as a nanoparticle or biotin. The phrases “pharmaceutical or pharmacologically acceptable” refers to molecular entities and compositions that do not produce an adverse, allergic or other untoward reaction when administered to an animal, such as, for example, a human, as appropriate.

As used herein, “pharmaceutically acceptable carrier” includes any and all solvents, dispersion media, coatings, surfactants, conjugates, antioxidants, preservatives (e.g., antibacterial agents, antifungal agents), isotonic agents, absorption delaying agents, salts, preservatives, drugs, drug stabilizers, gels, polymers, nanoparticles, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the pharmaceutical compositions is contemplated.

The agent(s) disclosed herein may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18^{th} Ed. Mack Printing Company, 1990, incorporated herein by reference).

The agent(s) disclosed herein may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

In some embodiments, the agent(s) disclosed herein are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, polymers, nanoparticles, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In some embodiments, the composition is combined with the carrier in any convenient and practical manner, i.e., by solution, suspension, emulsification, admixture, encapsulation, absorption and the like. Such procedures are routine for those skilled in the art.

In some embodiments, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In some embodiments, the composition is a pharmaceutical lipid vehicle composition that includes agent(s) disclosed herein, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the agent(s) disclosed herein may be dispersed in a solution containing a lipid, dissolved with a lipid, emulsified with a lipid, mixed with a lipid, combined with a lipid, covalently bonded to a lipid, contained as a suspension in a lipid, contained or complexed with or conjugated with a micelle or liposome, or otherwise associated with a lipid or lipid structure by any means known to those of ordinary skill in the art. The dispersion may or may not result in the formation of liposomes.

The actual dosage amount of a composition administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In some embodiments, the agent(s) disclosed herein are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the agent(s) disclosed herein may alternatively be incorporated with one or more excipients in the form of an orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In some embodiments, agent(s) disclosed herein may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In some embodiments, the active compound or STAT3 inhibitor may be formulated for administration via various miscellaneous routes, for example, topical or transdermal administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: STAT3 Suppresses the AMPK-ULK1-Dependent Induction of Autophagy in Glioblastoma Cells

### Introduction

Glioblastoma multiforme (GBM) is a highly aggressive and lethal brain tumor with a dismal prognosis (Shergalis A, et al. Pharmacol Rev. 2018 70(3):412-445). Despite recent advancements in the understanding of the pathobiology of GBM, the median survival of patients ranges between 10-15 months (Davis F G, et al. J Neurosurg. 1998 88(1):1-10; Stupp R, et al. N Engl J Med. 2005 352(10):987-96). GBM presents major treatment challenges due to its therapeutic resistance and accessibility, which is limited by the blood-brain barrier (MacDonald T J, et al. Neuro Oncol. 2011 13(10):1049-58). Thus, there is a compelling clinical need for a deeper understanding of GBM to develop new and targeted approaches to treatment.

Although GBM is characterized by marked intra-tumoral heterogeneity at both cellular and molecular levels, the PTEN/PI3K/Akt/mTOR signaling axis is a major factor of GBM biology (Chakravarti A, et al. J Clin Oncol. 2004 22(10):1926-33; Hu X, et al. Neoplasia. 2005 7(4):356-68; Akhavan D, et al. Neuro Oncol. 2010 12(8):882-9; Fan Q W, et al. Methods Mol Biol. 2012 821:349-59; Li X, et al. Oncotarget. 2016 7(22):33440-50; Mecca C, et al. Dis Markers. 2018 2018:9230479). In addition, a large body of evidence has shown that the transcription factor, Signal Transducer and Activator of Transcription 3 (STAT3), is an important oncogenic driver in many cancers including GBM (Brennan C W, et al. Cell. 2013 155(2):462-77; Gray G K, et al. Expert Rev Neurother. 2014 14(11):1293-306). Under normal physiological conditions, cytoplasmic STAT3 undergoes phosphorylation at both Tyrosine (Y)-705 and Serine (S)-727. Tyrosine phosphorylation of STAT3 induces homodimerization and/or heterodimerization with other STAT family proteins, nuclear translocation, and DNA binding, leading to the induction of cytokine responsive genes (Ihle J N. Curr Opin Cell Biol. 2001 13(2):211-7), and anti-apoptotic genes (Bhattacharya S, et al. Biochem J. 2005 392(Pt 2):335-44). The role of S727 phosphorylation is less well understood, but studies suggest that it may be required for STAT3's maximum transcriptional activity (Boulton T G, et al. Proc Natl Acad Sci USA. 1995 92(15):6915-9). STAT3 is constitutively phosphorylated in GBM cancer stem cells (GSCs) and inhibiting STAT3 phosphorylation attenuates GSC-driven tumor growth (Bu L L, et al. Oncotarget. 2015 6(39):41944-58; Ganguly D, et al. Oncotarget. 2018 9(31):22095-22112), showing that STAT3 plays a critical role in GBM tumorigenesis.

Phosphorylation-independent STAT3 pathways have also recently been identified. Upon cytokine treatment, the histone acetyl-transferase (HAT) CBP/p300 acetylates STAT3 on lysine, which enhances DNA binding, transactivation activity, and nuclear localization (Zhuang S. Cell Signal. 2013 25(9):1924-31). Conversely, histone deacetylases (HDACs) promote STAT3 deacetylation and inhibit transcription of STAT3 target genes (Yuan Z L, et al. Science. 2005 307(5707):269-73). Apart from acetylation, STAT3 function can be modulated through other post-translational modifications (PTMs), such as methylation. The histone methyltransferase, SET9, methylates promoter-bound STAT3 at Lys-140 in an IL-6-dependent manner in human colon cancer cells, which reduces STAT3 binding to DNA, and gene transcription (Yang J, et al. Proc Natl Acad Sci USA. 2010 107(50):21499-504). STAT3 can also be methylated at Lys-49 and Lys-180 by EZH2, the lysine methyltransferase subunit of the polycomb repressive complex 2 in GBM and breast cancer cell lines (Kim E, et al. Cancer Cell. 2013 23(6):839-52), increasing STAT3 transcriptional activity (Dasgupta M, et al. Proc Natl Acad Sci USA. 2015 112(13):3985-90). Although PTMs are critical for the pleiotropic functions of STAT3 (Yu H, et al. Nat Rev Cancer. 2014 14(11):736-46), it is unknown whether the crosstalk between STAT3 phosphorylation, acetylation, and methylation modifies GBM tumorigenesis and its therapeutic resistance.

Autophagy is a highly conserved cellular catabolic process that recycles damaged organelles, protein aggregates, and other toxic intracellular debris. Autophagy has a complex and context-dependent role in tumor development and cancer therapy (Levine B, et al. Cell. 2008 132(1):27-42; Mizushima N, et al. Nature. 2008 451(7182):1069-75; Galluzzi L, et al. EMBO J. 2015 34(7):856-80; Wen X, et al. Semin Cancer Biol. 2019 Nov. 7). Although autophagy has been found to suppress primary tumor growth (Degenhardt K, et al. Cancer Cell. 2006 10(1):51-64), it is required for advanced tumor growth with elevated metabolic demand and promotes multiple steps in the metastatic cascade (Mowers E E, et al. Oncogene. 2017 36(12):1619-1630). Constitutive activation of mTOR signaling impairs basal autophagy in GBM (Huang X, et al. J Clin Neurosci. 2010 17(12):1515-9), which enhances proliferation and pluripotency of GSCs (Jhanwar-Uniyal M, et al. Adv Biol Regul. 2013 53(2):202-10; Fu J, et al. Chin Med J (Engl). 2009 122(11):1255-9). Conversely, restoration of autophagy through mTOR inhibition reduces the invasive potential of GSCs (Chandrika G, et al. Sci Rep. 2016 6:22455; Catalano M, et al. Mol Oncol. 2015 9(8):1612-25), suggesting that mTOR hyperactivation sustains glioma cell metabolism through suppressing autophagy. Nonetheless, it is still unclear whether autophagy induction or inhibition represents the most promising approach for GBM treatment. Studies have associated increased autophagy with both tumor survival and chemoresistance in GBM (Fu J, et al. Chin Med J (Engl). 2009 122(11):1255-9; Shi F, et al. Neuroscience. 2017 346:298-308), and for the antitumor effects of Temozolomide (TMZ) combined with radiotherapy (Yan Y, et al. J Exp Clin Cancer Res. 2016 35:23).

Prominin-1 (Prom1/CD133) is well known as a cancer stem cell biomarker (Zhu L, et al. Nature. 2009 457(7229):603-7), but little is known about its role in GBM. Recent studies have implicated Prom1-mediated autophagy in cancer cell survival (Chen H, et al. PLoS One. 2013 8(2):e56878; Wei M F, et al. Autophagy. 2014 10(7):1179-92).

Genetic deletion of Prom1 activates both mTORC1 and mTORC2-dependent pathways causing inhibition of autophagy flux in human RPE cells (Bhattacharya S, et al. Invest Ophthalmol Vis Sci. 2017 58(4):2366-2387). In these studies, cytosolic Prom1 regulated autophagy flux by acting as a component of a molecular scaffold involving cytosolic p62 and HDAC6, and by regulating the trafficking of autophagosomes to lysosomes (Bhattacharya S, et al. Invest Ophthalmol Vis Sci. 2017 58(4):2366-2387).

Although these studies underscore the relevance of autophagy in GBM, little is known about the function of STAT3 signaling in autophagy regulation in GBM. Nuclear STAT3 inhibits autophagy through upregulating anti-autophagy genes and downregulating pro-autophagy genes (Yuan X, et al. Exp Cell Res. 2015 330(2):267-76). An inverse correlation between phosphorylated STAT3 and a stimulator of autophagy, Beclin1, has also been seen in GBM (Caldera V, et al. J Oncol. 2008 2008:219241). Based on these observations, experiments were conducted to define the specific STAT3-dependent signaling mechanisms that modulate autophagy and thereby, impact GBM tumorigenesis and chemosensitivity. Utilizing CRISPR/Cas9 knockout of STAT3 in established GBM lines and restoration with phosphorylation-defective mutants, aberrant STAT3 activation is demonstrated suppresses autophagy, which may be exploitable therapeutically.

### Materials and Methods

### Reagents

Materials purchased include the following: Fetal bovine serum (Atlanta Biologicals); Enhanced chemiluminescence (ECL) Western blot detection system (Perkin Elmer, Inc.); Protease/Phosphatase Inhibitor Cocktail, cleaved active caspase-3 (Asp 175); LC3-1/LC3-II; SQSTM1/p62, p-Akt Ser473, HDAC-6, phospho-S6 Ribosomal protein Ser235/236, p-STAT3 Y705, p-STAT3 Ser727, Acetyl-STAT3 Lys685, total-STAT3, p-AMPKα Thr172, Total-AMPKα, p-ULK1 Ser555, p-ULK1 Ser638, Total-ULK1, p-TSC2 Ser1387, p-TSC2 Ser1462, Total-Tuberin/TSC2, Beclin-1, BNIP3 and Cathepsin-D antibodies (Cell Signaling Technology, Inc.); Alexa-Fluor 488 conjugated, Alexa-Fluor 647, and Cy3 conjugated secondary antibodies (Molecular Probes); Anti-trimethyl STAT3 Lys180, and Bafilomycin A1, (EMD Biosciences/Millipore Corp.); ULK1 Inhibitor (MRT68921) (MedChemExpress). The ULK1 siRNA and transfection reagent were obtained from Santa Cruz Biotechnology. The cell death detection ELISA kit was purchased from Roche (Millipore Sigma). All chemicals were of the highest purity commercially available.

### Cell Culture

MT330 (UTHSC, Department of Neurosurgery) and LN229 (ATCC CRL-2611) were grown in DMEM containing high glucose, containing 10% fetal bovine serum, and supplemented with 1× antibiotic-antimycotic solution (Gibco, Thermo Fisher Scientific) at 37° C. with 5% CO₂, as described previously (Yang C H, et al. Cancer Lett. 2019 465:59-67). STAT3 was knocked out in both LN229 and MT330 cells by CRISPR/Cas9-mediated gene editing, and the constructs for wild-type (WT), Y705-STAT3 and S727A-STAT3 mutants were expressed in STAT3-KO cells by lentiviral transduction, as described previously (Ganguly D, et al. Oncotarget. 2018 9(31):22095-22112; Wang Y, et al. Cancer Lett. 2022 533:215614).

### Western Blotting

Cell lysates were prepared using mammalian protein extraction buffer (Cell Signaling Technology) and a protease inhibitor cocktail followed by SDS-PAGE. Proteins were transferred to Immobilon-P membranes (Millipore Bedford) and probed with primary antibodies overnight at 4° C. in TBS buffer containing 0.1% Tween-20 and 5% nonfat dry milk (Bio-Rad). Membranes were subsequently incubated with horseradish peroxidase-conjugated secondary antibodies at room temperature for 1 h, and the immunocomplexes were visualized by the ECL detection system (Perkin Elmer) quantified on the Azure Biosystems C500. Membranes were stripped and re-probed for actin or GAPDH as loading controls. Representative Western blots from three experiments are shown. Densitometric analysis of all Western blots was performed using Image J software.

### Immunoprecipitation

Glioblastoma cells were rinsed with ice cold PBS and lysed using a cell lysis buffer (Cell Signalling Technology, Inc.) containing protease and phosphatase inhibitors (Thermo Fisher Scientific). The lysates were clarified by centrifugation at 14,300 g for 15 min at 4° C. The cell extracts containing equal amounts of protein were incubated with STAT3 antibody overnight at 4° C. followed by addition of protein A/G agarose beads (Santa Cruz Biotechnology) with gentle rocking for 2 h. The beads were washed 3 times with lysis buffer and once with PBS, and the immunocomplexes were released by heating in Laemmli sample buffer and analyzed by Western blotting using trimethyl-STAT3 antibody (EMD Biosciences/Millipore Corp.).

### Immunofluorescence and Confocal Microscopy

Cells were cultured in chamber slides (Millipore) to ˜70% confluence and washed with PBS. Cells were fixed in 4% paraformaldehyde and methanol, and permeabilized with 1% Triton X-100. After blocking with 5% goat serum, cells were incubated with anti-rabbit LC3 and anti-mouse p62 antibodies and subsequently stained with Alexa Fluor 488 (goat anti-rabbit) and Alexa Fluor 633 (goat anti-mouse) secondary antibodies, as described previously. 24 DNA was counterstained with Vectashield mounting media with DAPI (Vectra Laboratories). Images were captured on a Zeiss LSM700 laser scanning confocal microscope.

siRNA Transfection

MT330 cells were grown to 60%-70% confluency in 6-well tissue culture plates, and siRNA transfection was performed using a protocol available from Santa Cruz Biotechnology. For ULK1 siRNA transfection, the cell monolayer was washed with siRNA transfection medium (Santa Cruz) and the siRNA/transfection reagent mixture was added dropwise on to the cell monolayer and incubated overnight at 37° C. in a CO₂ incubator. The following day complete growth medium containing 2 times the normal serum and antibiotics was added without removing the transfection mixture. After an additional incubation for 18-24 h, the medium was aspirated and replaced with fresh 1X growth medium. After another 24 h of incubation, cells were treated with or without 100 nM Baf and assayed for autophagy and apoptosis markers. Efficiency of transfection was monitored using FITC-conjugated control siRNA.

### Real-Time Quantitative PCR

TRIzol reagent (Thermo Fisher Scientific) was used to extract total-RNA. Total RNA concentrations were quantified by measuring A260 and A280 using NanoDrop spectrophotometry. Total-RNA (1 mg) was reverse-transcribed to cDNA using a kit from Promega and following manufacturer's instructions. The cDNA was diluted 1:5 with DNase-free water. Real-time qPCR was performed using an Ariamx Real-Time PCR system (Agilent Technologies) with 2.5 ml of the cDNA product in a 25 ml reaction mixture containing 1×SYBR® Green Master Mix (Applied Biosystems) and 120 nM forward and reverse primers. The qPCR conditions were 50° C. for 2 min, 95° C. for 10 min, followed by 40 cycles of 95° C. for 15 s and 60° C. for 1 min, as described previously. Samples were analyzed using the comparative ACT method, and gene expression was normalized to beta-actin expression. Data were presented as fold changes with empty-vector as control set to 1.0.

### Apoptosis

The quantitative DNA fragmentation assay was performed using a cell death ELISA kit as described earlier (Bhattacharya S, et al. Apoptosis. 2014 19(3):451-466). Briefly, MT330 cells were either treated with MRT68921 or ULK1 siRNA and the attached cells were washed twice with Dulbecco's phosphate buffered saline (DPBS). Cells were lysed, and an aliquot of the nuclei-free supernatant was placed in streptavidin-coated plates and incubated with anti-histone biotin and anti-DNA peroxidase-conjugated antibodies for 2 h at room temperature. After incubation, the samples were aspirated, and the wells were washed 3 times with incubation buffer. After the final wash, 100 μl of the substrate, 2,2′-azino-di[3-ethylbenzthiazolin-sulfonate], was added in the wells for 3 min at room temperature. The absorbance was read at 405 nm using the SpectraMax iD3 microplate reader (Molecular Devices). Results were expressed as absorbance at 405 nm/mg protein/min.

### Statistical Analysis

All data were analyzed by GraphPad Prism 9 program (GraphPad Software Inc.), and an unpaired 2-tailed Student's t-test was used to assess statistical significance. Data are expressed as mean±SE. Experiments were repeated three times, with triplicate samples for each. Unless otherwise stated, values of *p<0.05, **p<0.01, ***p<0.001 and ****p<0.0001 were considered significant.

### Results

### STAT3 Deletion Increases Autophagy

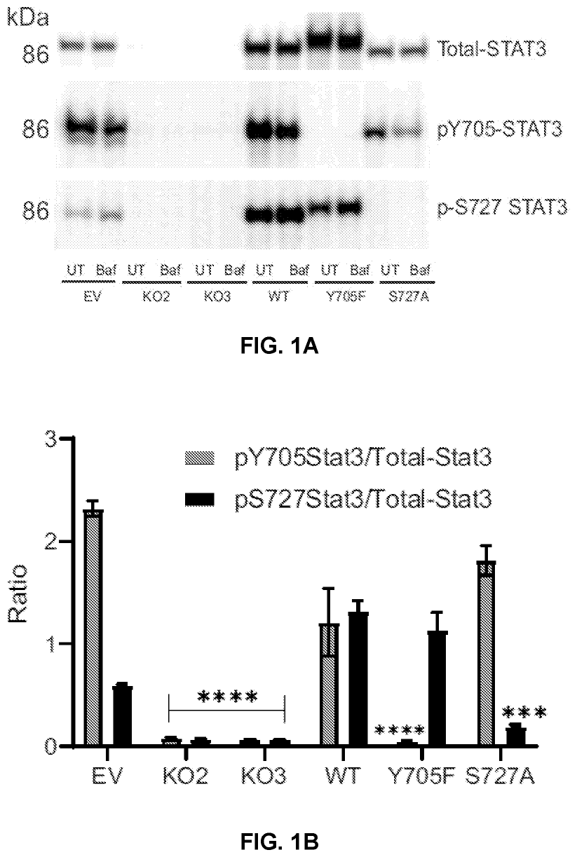

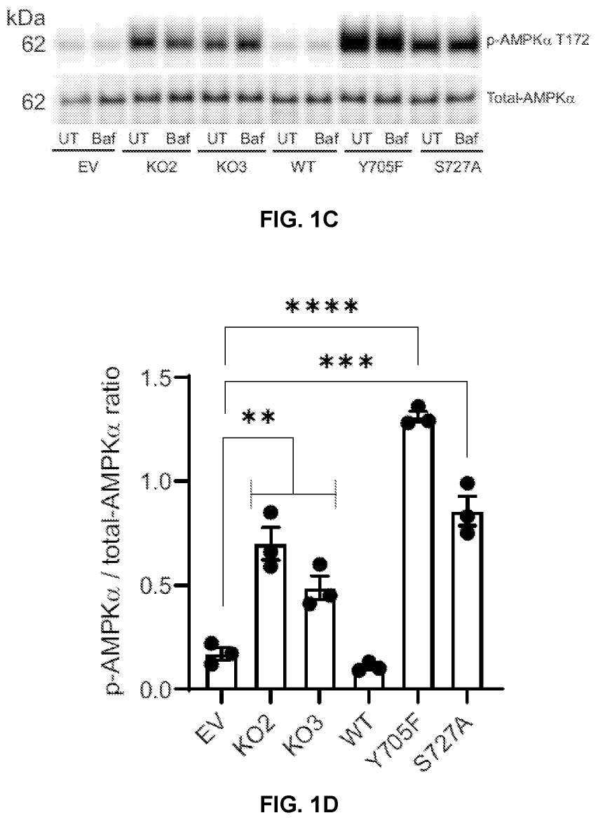

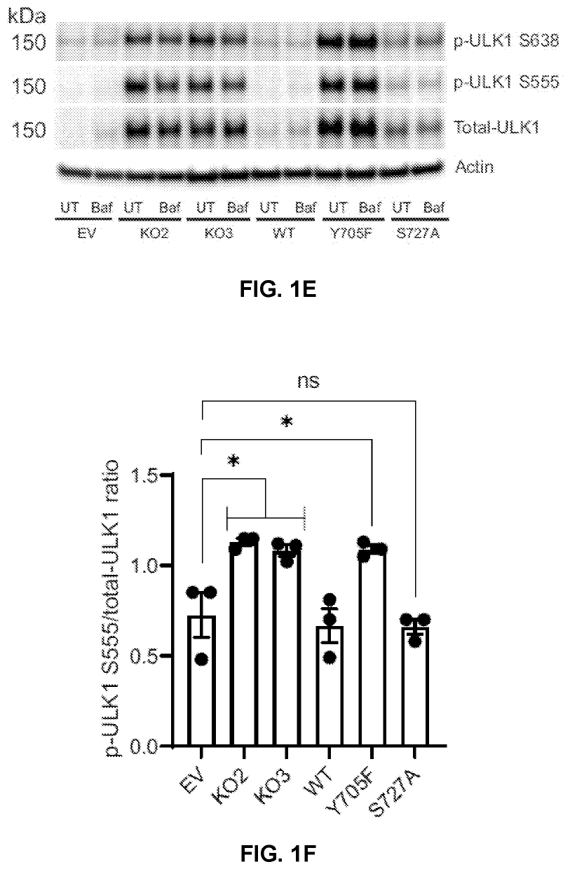

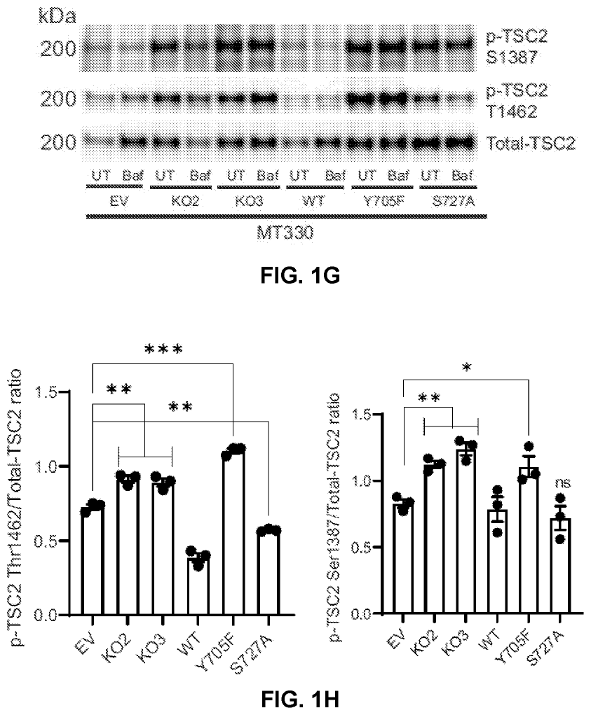

STAT3-KO MT330 GBM cells were previously established by CRISPR/Cas9 gene editing; as a control, cells were transduced with empty vector (EV). Absence of STAT3 protein in MT330 cells was validated in whole-cell extracts by immunoblotting with antibodies to STAT3 (FIG. 1A). STAT3 expression was restored in the STAT3 knockout cells by transduction with lentiviral vectors encoding either the WT-STAT3 or the STAT3 mutants (Y705F and S727A). In EV-transduced MT330 cells, STAT3 was phosphorylated on both Y705, and Ser727 (FIG. 1A,1B). While STAT3-KO completely abolished STAT3 serine and tyrosine phosphorylation, rescue of KO cells with WT-STAT3 restored these phosphorylation events. Expression of the Y705F mutant in KO cells restored S727 phosphorylation but showed no Y705 phosphorylation (FIG. 1B). Similarly, expression of the S727A mutant restored Y705 phosphorylation but S727 phosphorylation was blocked (FIG. 1B).

To characterize whether STAT3-deficiency may regulate autophagy in MT330 GBM cells, AMPKα and Unc-51-like kinase 1 (ULK1) phosphorylation status was examined in STAT3-KO MT330 cells. AMPKα is activated in response to cellular stress (Egan D, et al. Autophagy. 2011 7(6):643-644) and activates autophagy either through ULK1 or by impairing mTOR-dependent inhibition of ULK1 (Egan D, et al. Autophagy. 2011 7(6):643-644; Kim J, et al. Nat Cell Biol. 2011 13(2):132-141). Deletion of STAT3 in MT330 cells significantly increased AMPKα activity, as determined by the ratio of phosphorylated AMPKα (assessed by Thr172 phosphorylation in the catalytic domain) to unphosphorylated AMPKα (FIG. 1C,1D). Rescue of STAT3-KO cells with WT-STAT3 blocked AMPKα activation. Expression of the Y705F and S727A mutants in STAT3-KO cells had significantly higher levels of AMPKα activation compared to STAT3-KO cells (FIG. 1C,1D). Since AMPKα activates autophagy through ULK1 phosphorylation at multiple sites, ULK1 phosphorylation was next assessed at S555 and S638 residues (FIG. 1E,F). STAT3 deletion in MT330 cells markedly increased total ULK1 protein expression and phosphorylation at all sites (FIG. 1E). The results demonstrate significantly higher levels of phosphorylated ULK1 S555 in STAT3-KO cells compared to EV (FIG. 1F). Given that ULK1 acts as a direct target for both mTORC1 and AMPKα, 28 these data suggest that ULK1 is a critical mediator of autophagy in GBM cells. Expression of the Y705F mutant in KO cells showed a similar effect on phosphorylated ULK1 S555. However, cells expressing the S727A mutant had no significant effect on phosphorylated ULK1 (FIG. 1F) suggesting that S727-STAT3 phosphorylation is involved in AMPKα-dependent pathway but does not involve ULK1 to suppress autophagy. Reconstitution with WT-STAT3 in KO cells completely reversed both pathways. Bafilomycin (Baf) treatment had no effect on AMPKα and ULK1 phosphorylation in MT330 cells, indicating that these upstream pro-autophagy pathways are unaltered by disruption of autophagy flux.

Since AMPKα inhibits mTORC1 by phosphorylating and activating TSC2 (Shaw R J. Acta Physiol. 2009 196(1):65-80), TSC2 phosphorylation was examined in STAT3-KO MT330 cells. KO of STAT3 significantly increased TSC2 S1387 and Thr1462 phosphorylation, which was reversed with WT-STAT3 expression (FIG. 1G,1H). Expression of Y705F mutant also increased TSC2 S1387 and T1462 phosphorylation. In contrast, cells expressing the S727A mutant showed significantly lower levels of phosphorylated TSC2 T1462 but high levels of S1387 phosphorylation, demonstrating that STAT3 S727 phosphorylation selectively regulates TSC2 T1462 phosphorylation (FIG. 1G,1H). Thus, AMPKα activation in STAT3-KO cells acts through more than one pathway to activate autophagy, by directly activating ULK1 and by indirectly impairing inhibition of ULK1 through activation of TSC2 S1387 phosphorylation. Since Akt inhibits TSC2 via phosphorylation on T1462, resulting in basal mTORC1 activation, it is possible that mTORC1 could phosphorylate and inhibit ULK1. This may be counteracted by ULK1 phosphorylation at S555 leading to its sustained activity, which is required for maintaining autophagy flux in STAT3-KO cells.

### STAT3-Deletion Increases Autophagic Flux in LN229 Cells

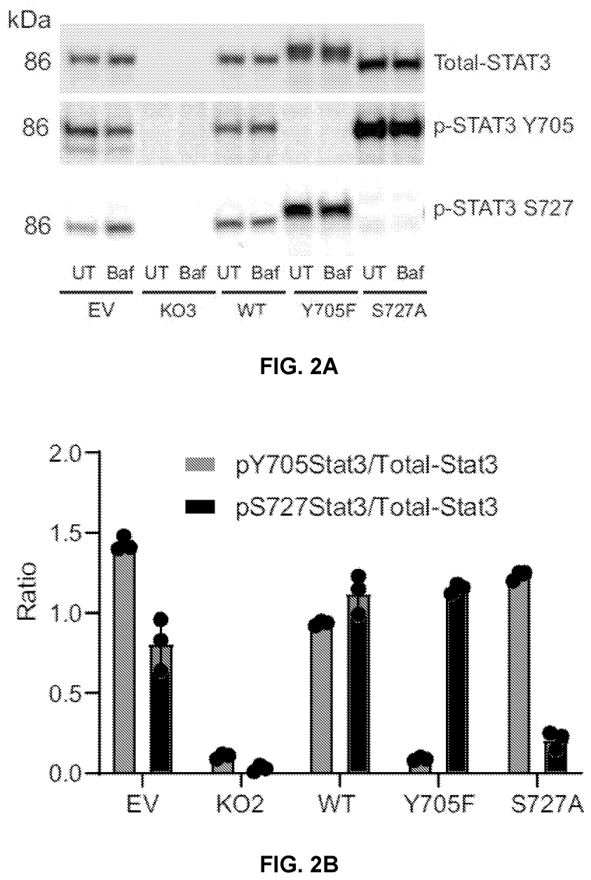

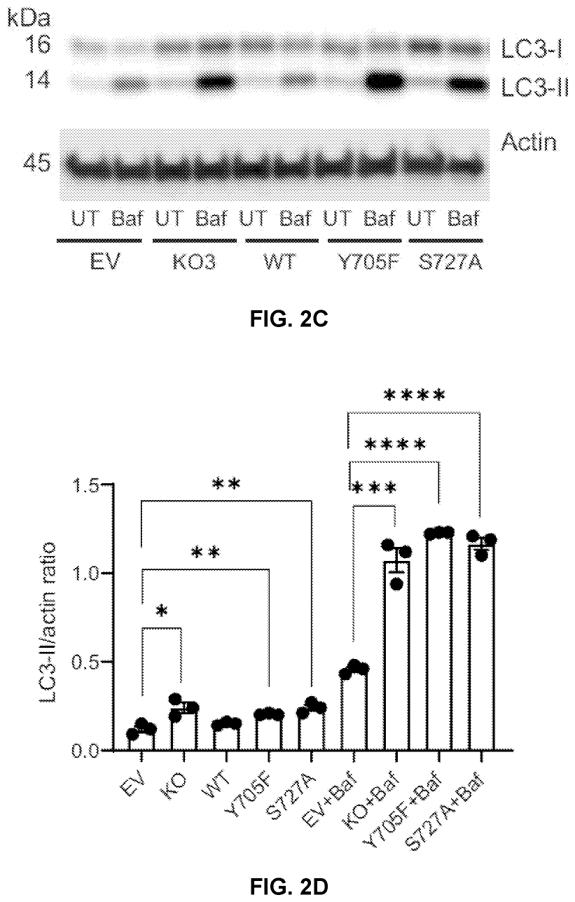

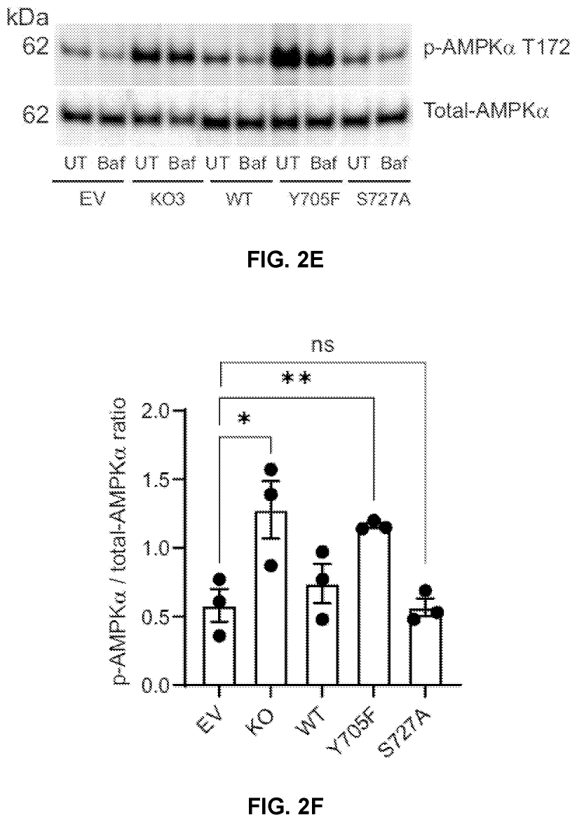

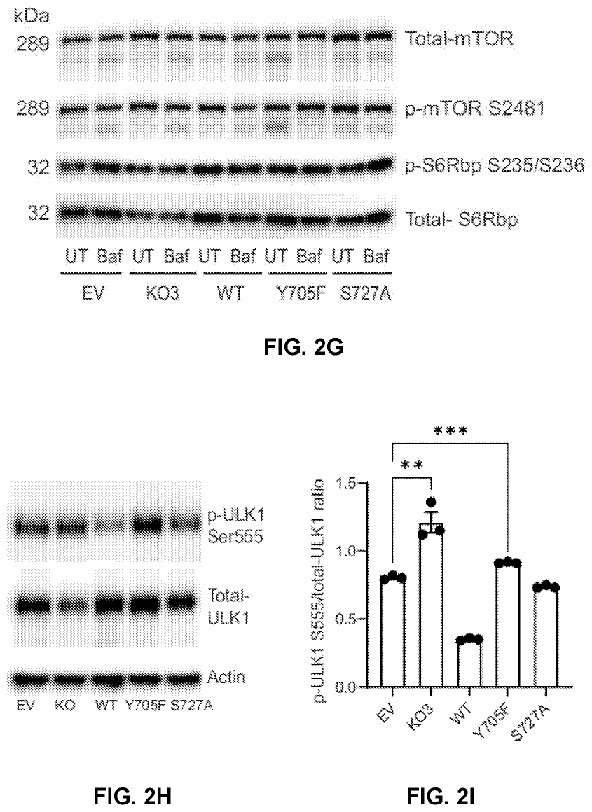

The role of STAT3 in GBM autophagy regulation was also examined in another GBM cell line, LN229 cells, in which STAT3 was also knocked out or restored with WT-STAT3 and STAT3 phosphodeficient mutants (Y705F and S727A). Consistent with the results in MT330 cells, STAT3 was phosphorylated on both Y705 and S727 sites in EV-transduced LN229 cells (FIG. 2A,B). Rescue with WT-STAT3 lentivirus in KO cells restored STAT3 expression and STAT3 Y705 and S727 phosphorylation. As with the MT330 studies, expression of the Y705F mutant eliminated Y705 phosphorylation and restored S727 phosphorylation (FIG. 2A,2B), and expression of the S727A mutant in STAT3-KO cells restored Y705 phosphorylation but not S727 phosphorylation (FIG. 2A,B).

To validate the findings on the role of STAT3 in autophagy, LN229 cells were treated with Baf and LC3-II levels determined. Baf is a specific inhibitor of vacuolar-type ATPase that is known to prevent the fusion of autophagosomes with lysosomes resulting in LC3-II accumulation and blockade of the autophagic flux (Yamamoto A, et al. Cell Struct Funct. 1998 23(1):33-42). Since autophagy is a dynamic process where microtubule-associated protein light chain-I (LC3-1, precursor) is rapidly converted to lipidated LC3-II, we examined the relative levels of LC3-1 and LC3-II in LN229 cells treated with Baf. While basal and Baf treated LN229 cells had low LC3-II levels, deletion of STAT3 significantly increased LC3-II levels, demonstrating constitutive autophagy upon STAT3 deletion (FIG. 2C,2D). Furthermore, rescue with WT STAT3 reduced the LC3-II levels, confirming that STAT3 suppresses autophagy in LN229 cells. As shown in FIG. 2C,2D, cells expressing the Y705F and S727A mutants showed high basal and Baf-induced LC3-II levels. These results suggest that both STAT3 serine and tyrosine phosphorylation events are necessary for suppressing autophagy in GBM cells, which is entirely consistent with findings in MT330 cells.

Deletion of STAT3 in LN229 cells increased AMPKα activity (assessed by T172 phosphorylation in the catalytic domain) (Hardie D G. Biochem Soc Trans. 2011 39(1):1-13), without altering total AMPKα protein levels (FIG. 2E) indicating higher AMPKα activation (FIG. 2F). While expression of the Y705F mutant in STAT3-KO cells also showed AMPKα activation, expression of S727A mutant did not alter AMPKα phosphorylation, supporting a critical role for Y705 STAT3 phosphorylation in suppressing autophagy.

The role of STAT3 in mTOR phosphorylation was also examined in LN229 cells. The mTOR protein and its basal phosphorylation status at S2481 were unaltered in the KO and lines expressing STAT3 phosphorylation-inactive mutants (FIG. 2G), demonstrating that basal mTOR activity is unaltered. Furthermore, expression of S6 ribosomal protein (S6Rbp) and its phosphorylation at S235/S236 were also unaltered in KO cells and cells expressing STAT3 mutants (FIG. 2G). Therefore, autophagy induction in LN229 cells either lacking STAT3 or expressing the phosphorylation-defective STAT3 mutants involves AMPKα but is independent of the mTOR pathway.

Next examined was whether STAT3-KO activates ULK1 signaling in LN229 cells. Data showed that ULK1 protein was detectable in LN229 cells. Although STAT3-KO decreased ULK1 protein levels (FIG. 2H), it significantly increased ULK1 activity (FIG. 2I). Rescue with WT-STAT3 decreased ULK1 activity (FIG. 2H,2I), but expression of the Y705 mutant increased ULK1 activity in LN229 cells, which is consistent with our findings in MT330 cells. However, expression of the S727A mutant in STAT3-KO cells had no effect on ULK1 activity, also suggesting that the Y705 mutant is the key critical regulator of AMPKα and ULK1 signaling in LN229 cells.

### The Dependence of Autophagy on STAT3

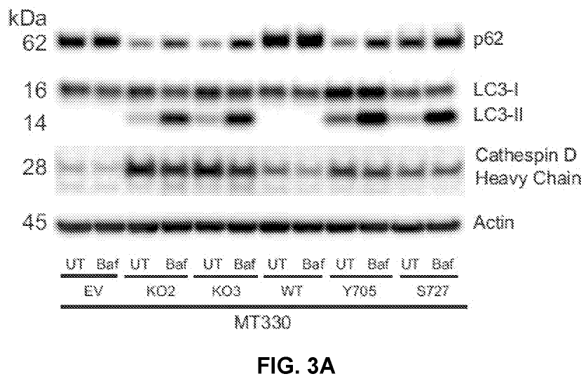

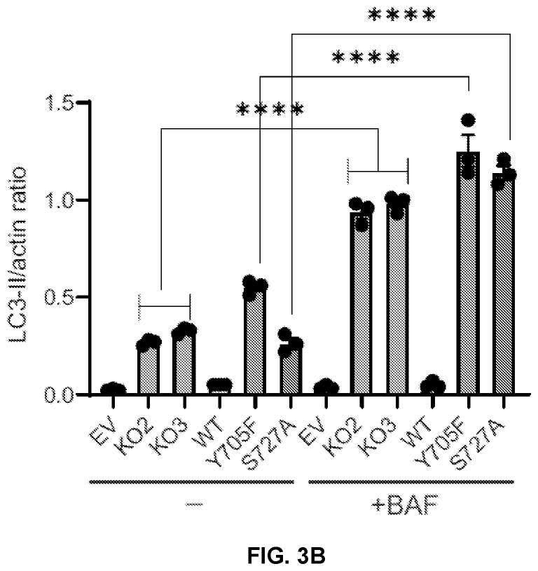

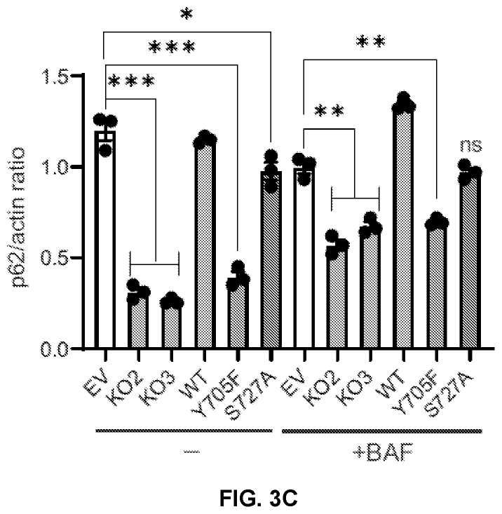

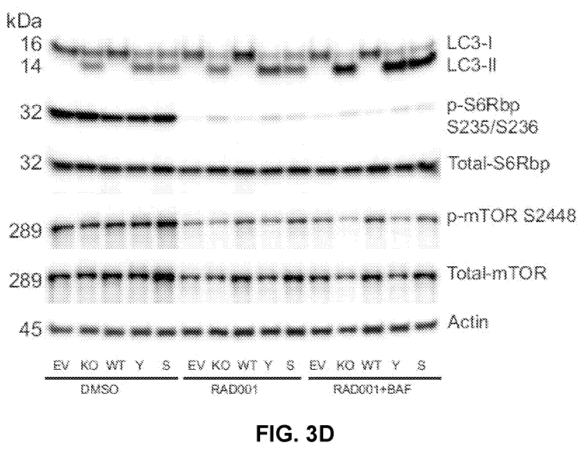

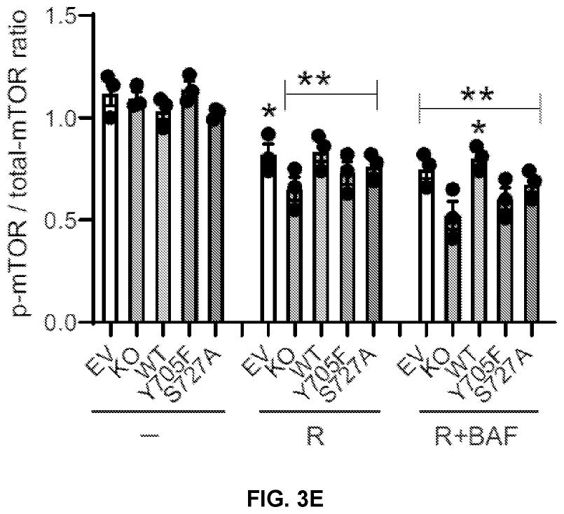

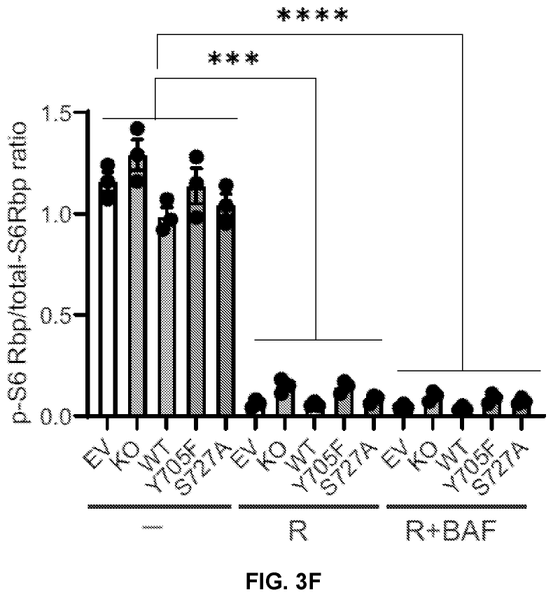

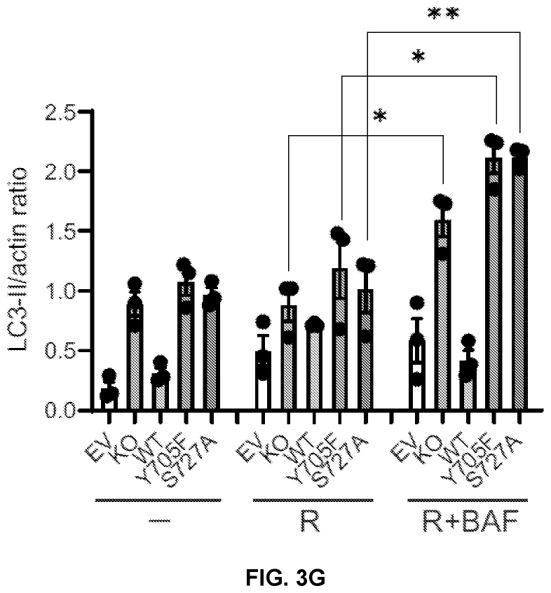

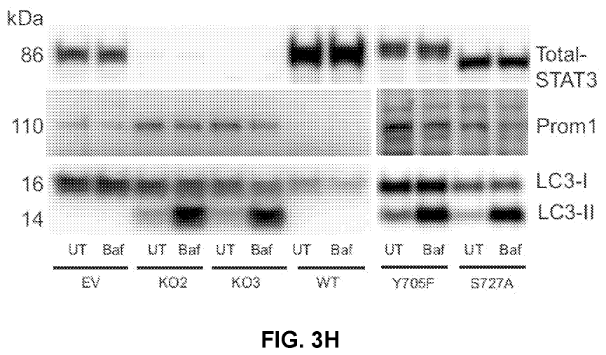

To further assess the impact of STAT3 deletion and rescue of KO cells with STAT3 mutants, MT330 cells were treated with Baf. Under basal conditions, LC3-II levels were undetectable in control MT330 EV cells and Baf treatment had no effect on LC3-II levels, showing minimal basal autophagy (FIG. 3A,B). In contrast, STAT3 deletion in MT330 cells significantly increased basal LC3-II, which was further increased by Baf treatment. Rescue with WT STAT3 ablated basal and Baf-induced LC3-II levels (FIG. 3A,B). These results demonstrate that KO of STAT3 enhances LC3-II levels and autophagy flux, which were reversed by WT-STAT3 restoration. Expression of Y705F and S727A phosphorylation-defective mutants also resulted in high basal and Baf-induced LC3-II levels as observed in STAT3-KO cells, providing further evidence for the role of these phosphorylation events in STAT3 autophagy suppression. Together, these results show that loss of STAT3 function enhances LC3-II lipidation and autophagic flux through activation of the AMPKα/ULK1/TSC2 signaling axis.

The p62 protein has often been used as an inverse marker for autophagy flux, and high expression of p62 is found in GBM patient tumors (Galavotti S, et al. Oncogene. 2013 32(6):699-712). High p62 levels were observed in control MT330 (EV) cells, but p62 levels were significantly decreased in STAT3-KO cells consistent with induction of autophagy in these cell lines (FIG. 3A,3C). Baf treatment partially increased p62 accumulation in STAT3-KO cells suggesting impaired autophagosomal degradation. Rescue of KO cells with WT-STAT3 restored p62 accumulation to that of control EV cells demonstrating the role of STAT3 in suppressing autophagy. Expression of Y705F mutant significantly decreased p62 accumulation, which is evidence of increased autophagosomal degradation and re-activation of autophagy (FIG. 3A,3C). Similarly, expression of the S727 mutant decreased p62 accumulation, suggesting a decline in its autophagosomal degradation. Conversely, Baf treatment did not significantly change LC3-II levels in cells expressing the S727A mutant. Our results, therefore, strengthen the hypothesis that Y705 STAT3 plays a key role in suppressing autophagy.

Additional evidence for the induction of autophagy was seen by an increase in the levels of mature heavy chain Cathepsin-D in STAT3-KO cells, which was blocked when KO cells were rescued with WT-STAT3 (FIG. 3A). In cells expressing phosphorylation-defective STAT3 mutants, the levels of mature Cathepsin D were higher compared to control EV cells (FIG. 3A). Cathepsins are lysosomal proteases that are essential for the breakdown of the recycled cellular components sequestered by the autophagosomes (Uchiyama Y. Arch Histol Cytol. 2001 64(3):233-246). Increase in the levels of lysosomal proteases upon deletion of STAT3 is consistent with autophagosome-fusion and autophagy activation.

A catalytic mTOR inhibitor, Everolimus (RAD001), was used to further characterize the role of STAT3 in regulating autophagy by mTORC1. Treatment of EV MT330 cells with Everolimus inhibited both mTOR S2448 phosphorylation (FIG. 3D,3E) and pS6K phosphorylation (a downstream target of mTORC1) (FIG. 3D,3F) but had no significant effect on autophagy induction (FIG. 3D,3G). STAT3 deletion had no effect on basal mTOR S2448 and S6Rbp phosphorylation S235/236 (FIG. 3D-3F) indicating that STAT3 does not regulate mTORC1 activity in MT330 cells. LC3-II levels in STAT3-KO cells significantly increased in response to Everolimus and Baf compared to Everolimus alone, demonstrating that combining STAT3 deletion with mTOR inhibition increases autophagy (FIG. 3D,G). These effects were reversed in cells reconstituted with WT STAT3. Expression of either Y705F or S727A mutants (as with the STAT3-KO cells) significantly increased LC3-II/actin ratio in the presence of Everolimus and Baf (FIG. 3D,3G). These results demonstrate that Everolimus enhances autophagic flux in cells lacking STAT3 and STAT3-KO cells expressing STAT3 phosphodeficient mutants.

Since Prom1 is a pro-autophagy protein in normal cells (Bhattacharya S, et al. Invest Ophthalmol Vis Sci. 2017; 58(4):2366-2387), Prom1 expression and its correlation with autophagy induction was examined in GBM cells. STAT3-KO increased Prom1 expression, which was reversed by rescue with WT-STAT3 in MT330 cells (FIG. 3H). Expression of Y705F and S727 mutants in STAT3-KO MT330 cells also showed increased Prom1 expression, which correlated with high autophagy (FIG. 3H). These results suggest that the induction of autophagy in cells lacking STAT3 is in part dependent on Prom1 expression. Reconstitution of WT-STAT3 in these cells completely reversed these effects confirming STAT3-mediated suppression of autophagy.

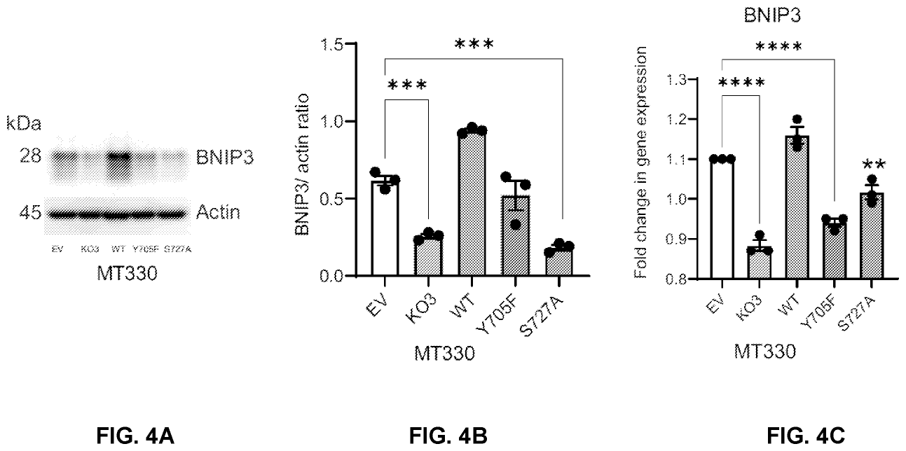

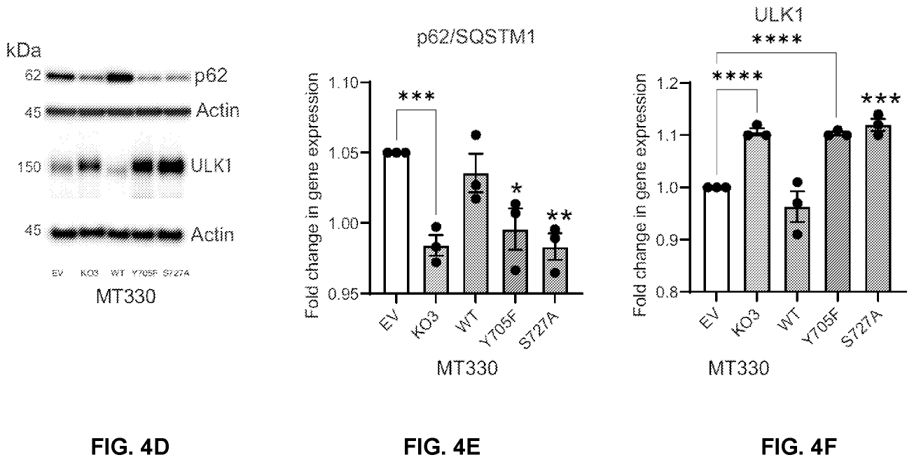

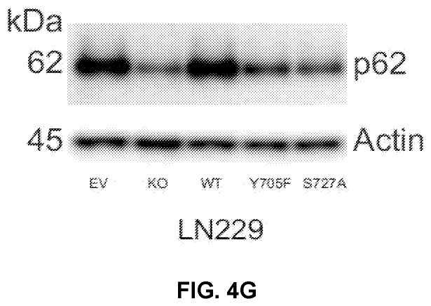

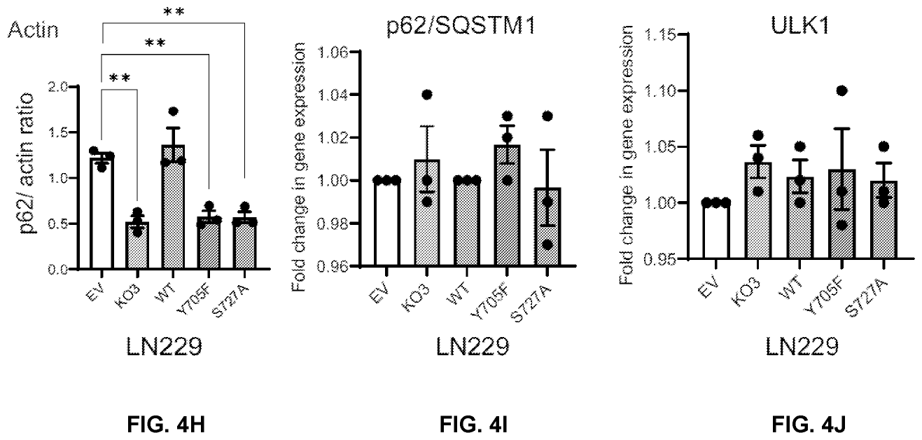

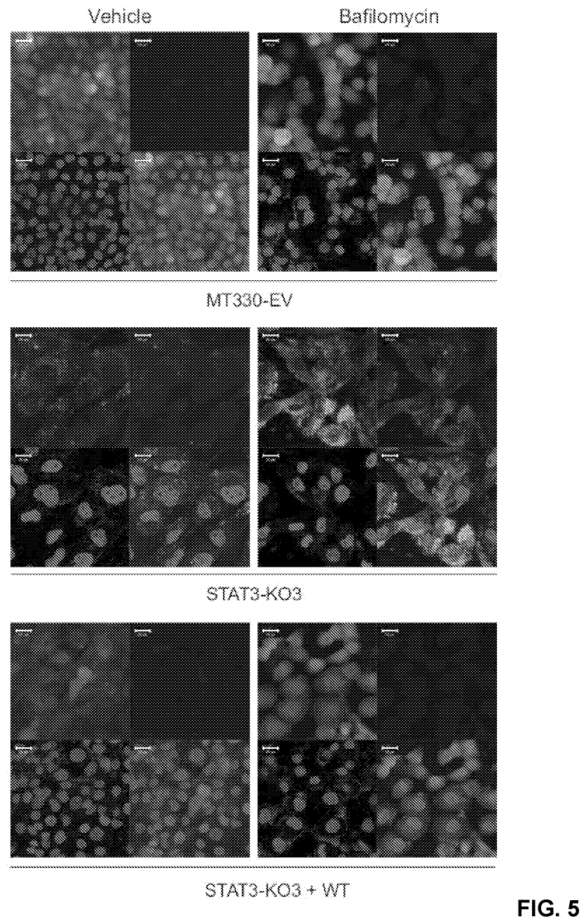

The role of STAT3 on autophagy-associated gene expression BNIP3 has been shown to play a role in regulating the autophagy pathway (Quinsay M N, et al. Autophagy. 2010 6(7):855-862). In previous studies, it was shown that STAT3-KO in MT330 cells inhibited the expression of classical STAT3 genes such as Cyclin D1 and vascular endothelial growth factor (Wang Y, et al. Cancer Lett. 2022 533:215614). STAT3-KO decreased BNIP3 protein levels in MT330 cells (FIG. 4A,B). Expression of Y705F and S727A mutants decreased BNIP3 protein levels (FIG. 4A) and WT-STAT3 reversed these changes. Since STAT3-KO increased ULK1 protein levels and decreased p62 levels in MT330 cells (FIGS. 1 and 3), qPCR was next performed on these autophagy-related genes. In agreement with these observations, our data show that STAT3 increases BNIP3 gene expression in MT330 cells (FIG. 4C), showing a correlation between mRNA and protein levels. Although BNIP3 is a potential target of ULK1, decreased BNIP3 protein levels in STAT3-KO and mutant expressing cells indicate that BNIP3 is degraded by ULK1-dependent autophagy, a mechanism found to decrease BNIP3 protein levels in tumor cells (Park C W, et al. Autophagy. 2013 9(3):345-360).

Consistent with data in FIG. 3, STAT3-KO reduced p62 protein and transcript levels (FIG. 4D,4E), which is reversed by WT-STAT3 expression. In addition, both Y705 and S727 residues are necessary for expression of p62 transcript and protein levels since cells expressing STAT3 mutants showed similar expression of p62 to STAT3-KO cells. In contrast, STAT3 reduces expression of ULK1 gene (FIG. 4F) and STAT3 phosphorylation on both Y705 and S727 residues is necessary for ULK1 repression (FIG. 4D,4F). Together, these results demonstrate that STAT3 may regulate autophagy in MT330 cells in part through the transcriptional regulation of several autophagy-related genes.

p62 and ULK1 transcript levels were next examined in LN229 cells. The data demonstrated that STAT3-KO and expression of STAT3 mutants significantly reduced p62 protein levels (FIG. 4G,H). To demonstrate whether mRNA and protein levels of p62 and ULK1 were correlated, their transcript levels were examined in LN229 cells. p62 and ULK1 gene expressions were unaltered in STAT3-KO LN229 cells (FIG. 4I,4J), which differed from their expression in STAT3-KO MT330 cells. In addition, expression of STAT3 mutants in STAT3-KO cells had no effect on p62 and ULK1 genes. These results demonstrate that reduction of p62 protein levels in LN229 cells is due to enhanced degradation during autophagy, not due to changes in p62 gene transcription.

### Immunolocalization of LC3 and p62

To further validate the role of STAT3 in autophagy, immunolocalization studies were performed to detect LC3 and p62 puncta formation. Under basal conditions, diffuse LC3 (green) and p62 (red) staining (green) was observed predominantly in the cytoplasm of control (EV) MT330 cells with some nuclear staining (FIG. 5). Treatment of EV cells with a low dose of Baf (1 μM) for 48 h had no effect on LC3 and p62 staining. These observations are consistent with data presented in FIG. 3 and demonstrate impaired autophagy in MT330 cells. Conversely, STAT3-KO cells without bafilomycin treatment showed distinct LC3 and p62 puncta formation, and treatment with bafilomycin for 48 h increased both LC3+ and p62 puncta formation and their co-localization (FIG. 5). Reconstitution of WT-STAT3 in STAT3-deleted cells completely reversed these effects. These observations confirm cellular induction of autophagy flux in response to STAT3 deletion.

### Targeting of ULK1 Blocks Autophagy and Induces Apoptosis in STAT3-KO MT330 Cells

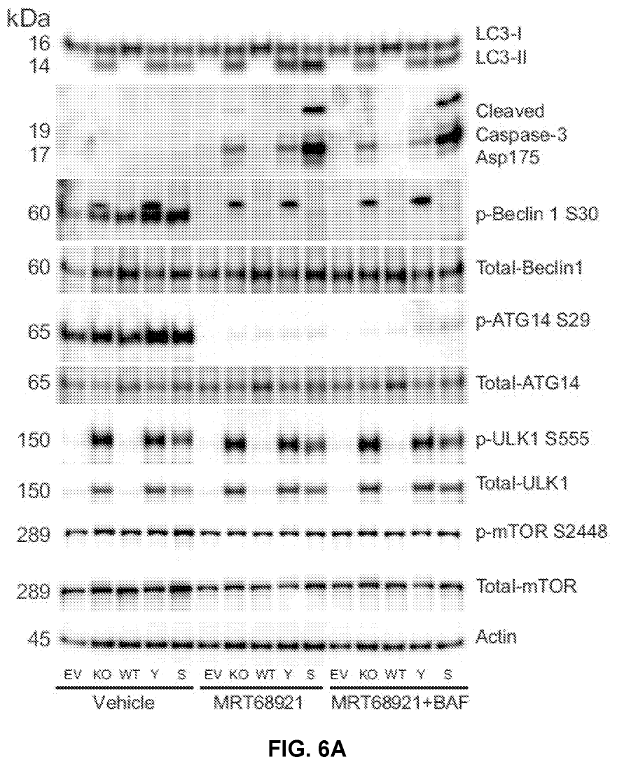

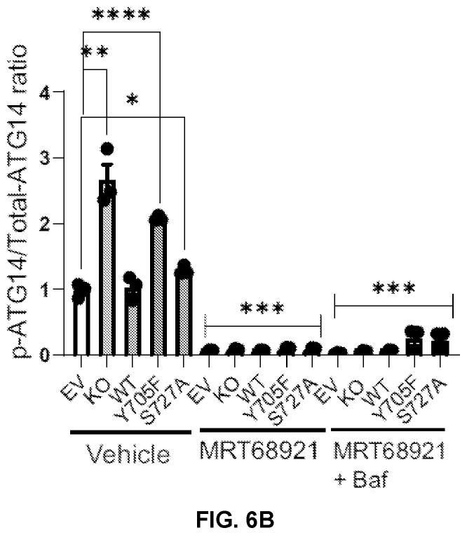

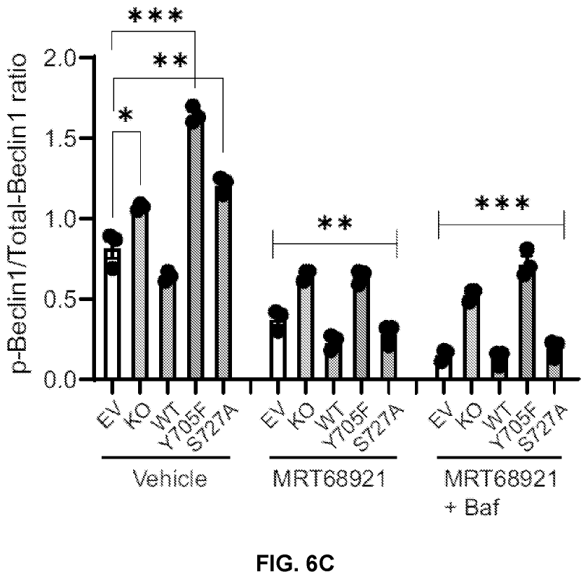

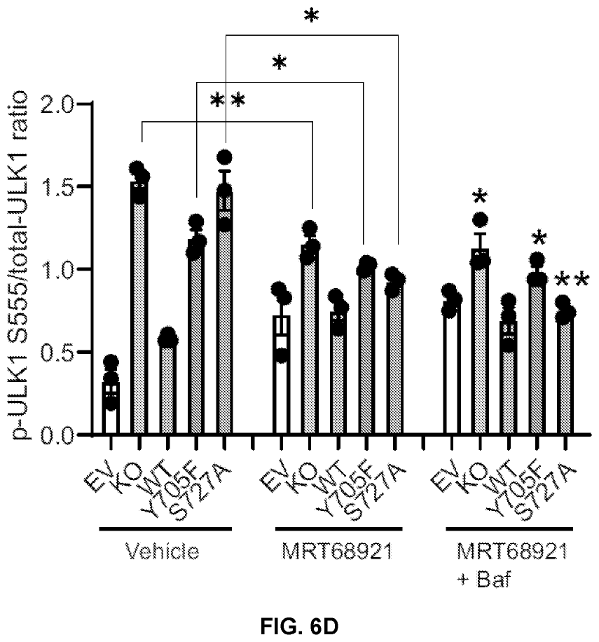

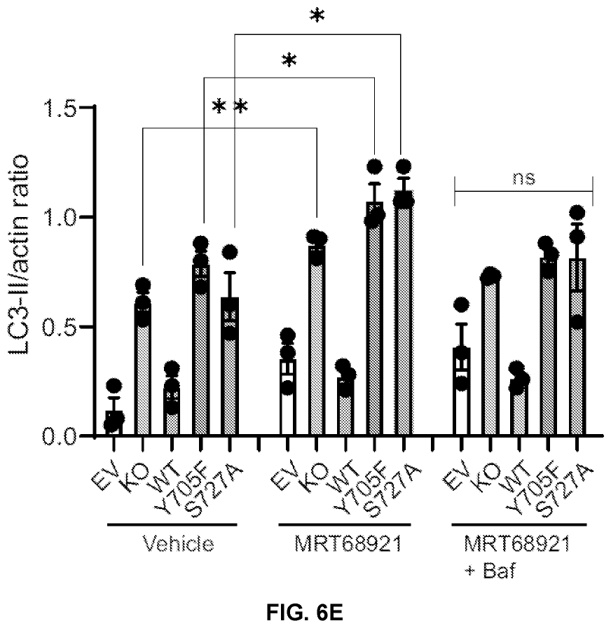

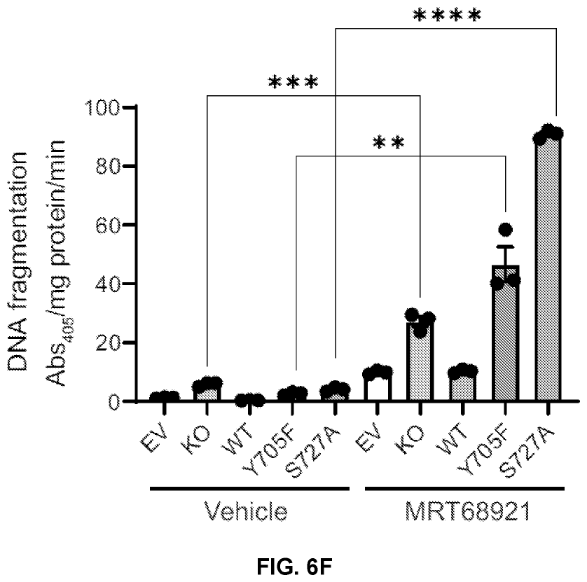

Because STAT3-KO in MT330 cells induces autophagy and concomitantly activates AMPKα/ULK1 signaling, the effects of pharmacologic inhibitors of ULK1 were tested. Previous studies showed that MRT68921 (inhibitor of ULK1 and ULK2) blocks autophagy flux (Petherick K J, et al. J Biol Chem. 2015 290(18):11376-11383; Follo C, et al. Mol Carcinog. 2018 57(3):319-332), demonstrating the importance of ULK kinase activity in autophagy. Since activated ULK1 phosphorylates both ATG14 on S29 and BECN1 on S30 for autophagy (Park J M, et al. Autophagy. 2018 14(4):584-597), ATG14 and BECN1 phosphorylation in response to MRT68921 treatment was measured. STAT3-KO cells and cells expressing STAT3 mutants had elevated levels of ATG14 S29 phosphorylation and Beclin1 S30 phosphorylation (FIG. 6A-6C). MRT68921 completely blocked ATG14 S29 phosphorylation demonstrating the efficacy of this inhibitor in targeting ULK1 and showing that constitutive activation of ULK1 in STAT3-KO and STAT3-mutant expressing lines leads to increased ATG14 phosphorylation on S29 (FIG. 6B). MRT68921 also reduced Beclin1 phosphorylation (FIG. 6C), demonstrating that ULK1 regulates Beclin1 activation in STAT3-KO and STAT3-mutant expressing lines. Importantly, MRT68921 significantly inhibited the high levels of ULK1 phosphorylation in STAT3-KO cells as well as cells expressing STAT3 mutants (FIG. 6D) confirming that Atg14 and Beclin1 are downstream targets of ULK1 signaling in MT330 cells. MRT68921 did not significantly block mTOR S2448 phosphorylation (FIG. 6A). Since mTORC1 is phosphorylated predominantly on S2448, these observations suggest that ULK1 inhibition has no significant effect on mTORC1.

MRT68921 also significantly increased LC3-II levels in STAT3-KO cells and in STAT3-KO cells transduced with the phosphorylation-defective Y705F or S727A mutants (FIG. 6A,6E). Given that ULK1 inhibition has been shown to disrupt autophagosome maturation downstream of LC3 conjugation (Petherick K J, et al. J Biol Chem. 2015 290(18):11376-11383), the increase in LC3-II levels in MRT68921-treated cells reflects a decrease of LC3 autolysosomal degradation. Treatment of cells with MRT68921 and Baf did not increase LC3-II levels as compared to DMSO-treated controls, showing inhibition of autophagy flux. Taken together, these results strongly suggest that MRT68921 blocks autophagy flux in cells lacking functional STAT3.

To further elucidate the role of ULK1 inhibition in MT330 cells, the effect of MRT68921 treatment on apoptosis we determined by caspase-3 cleavage. MRT68921 induced apoptosis in STAT3-KO and STAT3-mutant expressing lines but not in control EV and cells reconstituted with WT-STAT3 (FIG. 6A). Interestingly, STAT3-KO cells expressing the S727A-STAT3 mutant showed high levels of active caspase-3 suggesting increased a critical role of Y705 STAT3 phosphorylation in the sensitivity of GBM cells to MRT68921-induced apoptosis. To confirm apoptosis induction by MRT68921, a highly sensitive ELISA was used to quantify apoptotic cell death. MRT68921 significantly increased apoptosis in STAT3-KO and mutant expressing cells when compared to control cells (FIG. 6F). The extent of apoptotic cell death was considerably higher in cells expressing the S727A-STAT3 mutant (FIG. 6F), which is consistent with the levels of active caspase 3 in response to MRT68921 treatment (FIG. 6A). These observations demonstrate that autophagy can have a cytoprotective role in STAT3-KO and STAT3-mutant expressing lines, but apoptosis was rapidly induced by the addition of the ULK1-dependent autophagy inhibitor MRT68921.

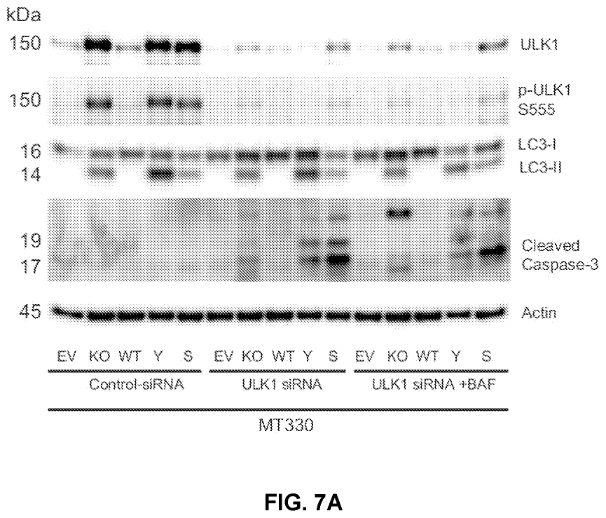

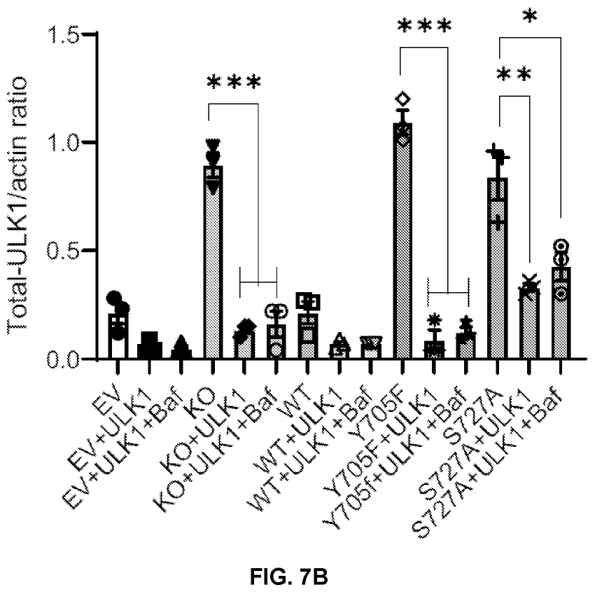

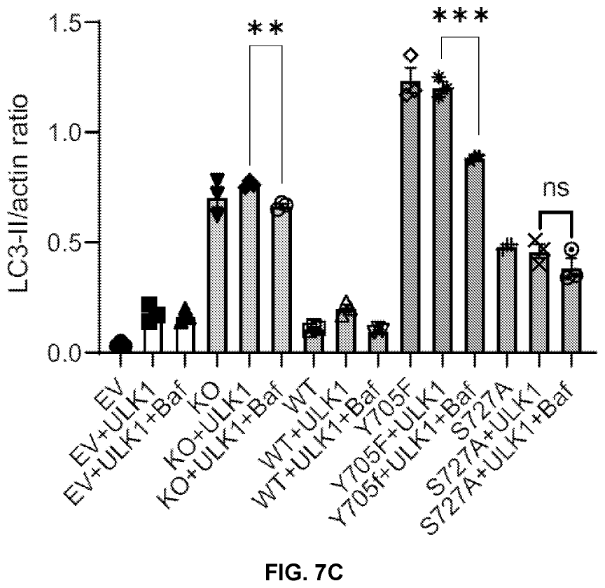

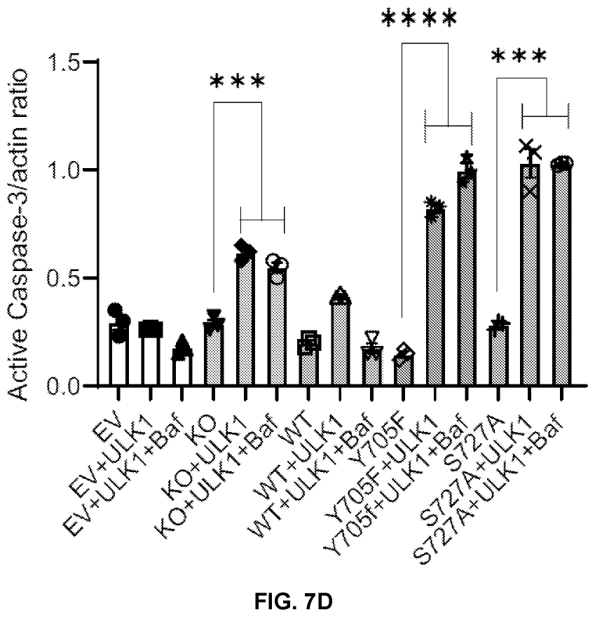

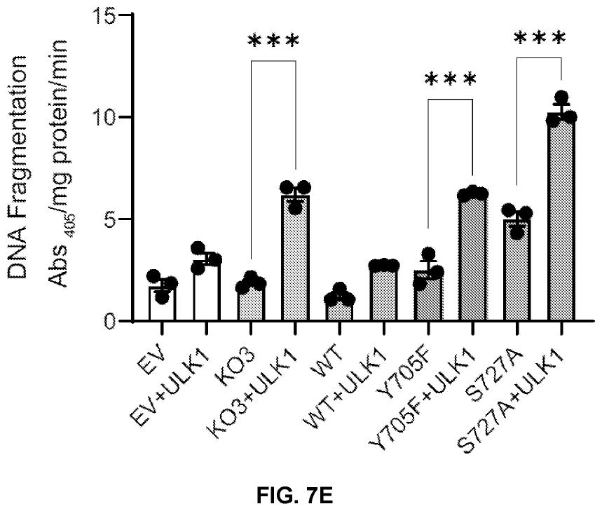

To further confirm the role of ULK1 in autophagy regulation, ULK1 was knocked down with siRNA in STAT3-deleted MT330 cells and STAT3-mutant expressing lines that contained high levels of ULK1 (FIG. 1E). Control scrambled siRNA had no effect on ULK1 levels, but ULK1-specific siRNA significantly decreased ULK1 levels in STAT3-KO cells and cells expressing both Y705F and S727A mutants (FIG. 7A,7B). LC3-II levels did not increase in ULK1 knockdown cells but significantly decreased in knockdown cells treated with Baf (FIG. 7A,C). However, LC3-II levels remained unchanged in ULK1 knockdown cells expressing the S727A mutant and treated with ULK1-siRNA (FIG. 7C), again demonstrating a key role of Y705 phosphorylation in the STAT3-mediated suppression of autophagy via ULK1 signaling. These studies suggest that autophagy induction by combining both STAT3 and ULK1 inhibition may be therapeutically beneficial for autophagy defective GBM cells.

To investigate whether ULK1 knockdown induces apoptosis, caspase-3 activation was measured. Consistent with observations involving inhibiting ULK1 activity by MRT68921 (FIG. 6), ULK1 knockdown increased caspase-3 activation and cell death in STAT3-KO cells and cells expressing both phosphorylation-defective mutants in the presence and absence of Baf (FIG. 7A,D,E). However, caspase-3 cleavage and cell death were considerably greater in cells expressing the S727A-STAT3 mutant treated with ULK1-siRNA.

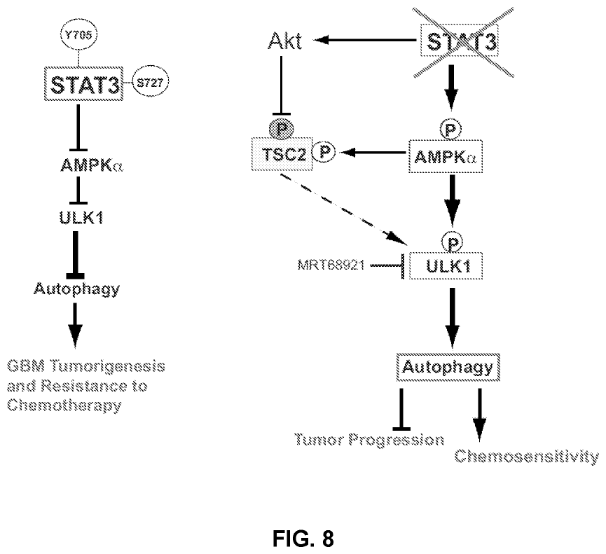

Based on the data, a conceptual model (FIG. 8, left) is proposed by which STAT3 represses autophagy and promotes GBM tumorigenesis. STAT3's post-translational modifications (PTMs) are responsible for inhibiting autophagy in GBM cells. STAT3 PTMs inhibit AMPKα and ULK1 signaling in GBM cells, which in turn inhibit autophagy. This promotes GBM tumor formation and increases chemoresistance. OData indicated that both Y705 and S727 phosphorylation are essential for autophagy suppression, and Y705 plays the predominant role in the inhibition of AMPKα/ULK1 signaling. Deleting STAT3 decreases p62 protein levels, increases AMPKα activity, and ULK1 gene expression and activity (FIG. 8, right). These changes in cellular signaling trigger autophagy in GBM cells. Under basal conditions, mTOR is activated in GBM cells, but autophagy induction in STAT3-KO cells is independent of the mTOR pathway.

Activated AMPKα phosphorylates TSC2 at S1387, which keeps mTORC1 activity in check leading to autophagy. Because STAT3 deletion activates Akt at S473, which inhibits TCS2 via phosphorylation at T1462, resulting in basal mTORC1 activation, it was speculated that baseline activation of mTORC1 in GBM cells could phosphorylate and inhibit ULK1 at S638. This is counteracted by extensive ULK1 phosphorylation at multiple sites causing its sustained activation and favoring its association with AMPKα. As a result, AMPKα directly phosphorylates ULK1 at S555 maintaining autophagy flux in STAT3-KO cells.

STAT3 deletion also increases expression of the pro-autophagy protein, Prom1 (Bhattacharya S, et al. Invest Ophthalmol Vis Sci. 2017; 58(4):2366-2387), in GBM cells. These molecular events enhance autophagy flux and reduce GBM tumorigenesis. Inhibiting ULK1 signaling in STAT3-KO cells validates the conceptual model described above. Inhibition of mTORC1 by Everolimus stimulates autophagy, whereas inhibition of ULK1 (by MTY68921 and siRNA) inhibits autophagy and induces apoptosis in STAT3-KO cells. Together, these studies demonstrate that STAT3-dependent suppression of autophagy is an essential contributor to GBM biology and that restoration of autophagy by a combined approach through STAT3 inhibition and AMPKα/ULK1 activation may be a novel approach to treat and overcome chemoresistance in GBM.

### Discussion

A key finding of these studies is that constitutive STAT3 phosphorylation suppresses autophagy in GBM cells. KO of STAT3 and the expression of STAT3 phosphorylation-defective mutants in GBM cells increase autophagy via upregulation of p62/SQSTM1 degradation, LC3 conversion and lysosomal activity. Loss of STAT3 functional activity increases phosphorylation of AMPKα, TSC2 and ULK1 on multiple sites, which were reversed by expression of ectopic WT-STAT3 in STAT3-KO GBM cells. Expression of the Y705F and S727A mutants in STAT3-KO LN229 and MT330 cells robustly increases autophagy, demonstrating that STAT3 function is indispensable for suppressing autophagy in GBM cells.

The role of autophagy in cancer is controversial because it has been reported to both promote and inhibit tumorigenesis (Marinkovic M, et al. Oxidative Med Cell Longev. 2018 2018:8023821). Autophagy confers drug resistance to radiotherapy and chemotherapy but also slows tumor progression (Galluzzi L, et al. Nat Rev Drug Discov. 2017 16(7):487-511). In contrast, rapid tumor growth in GBM and insufficient nutrient supply from the tumor vasculature contribute to activating autophagy and desensitizing tumor cells to chemotherapy (Ryskalin L, et al. Int J Mol Sci. 2019 20(15):3824). Basal autophagy was undetectable in MT330 GBM cells and that STAT3-KO induced autophagy without altering mTOR activity in both MT330 and LN229 cells. Inhibiting mTOR in STAT3-KO MT330 cells potentiated autophagy suggesting that combining STAT3 and mTOR inhibitors may improve GBM responses to treatment. Given these complex relationships, a better understanding of autophagy induction in response to STAT3 inhibition will benefit our understanding of GBM chemoresistance.

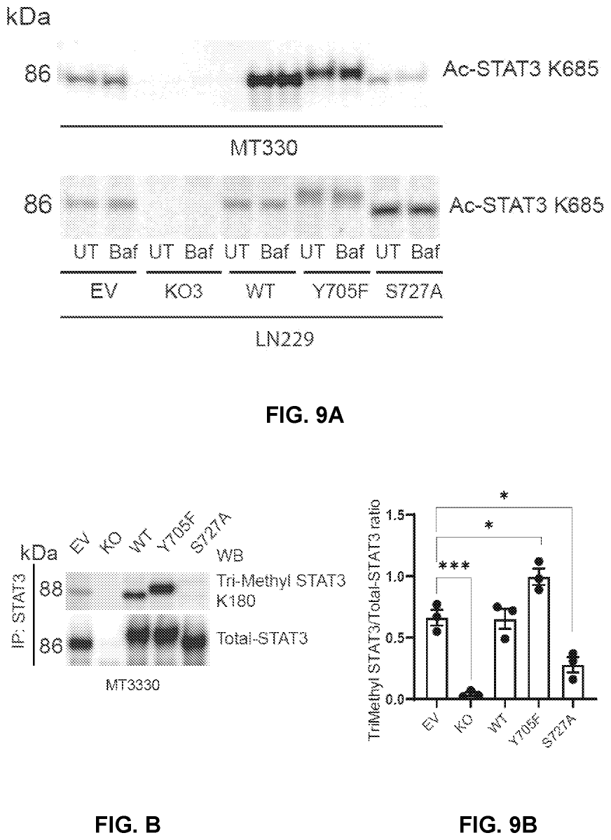

STAT3 undergoes serine and tyrosine phosphorylation that are molecular switches governing STAT3 activation and localization. Besides its well-known Y705 phosphorylation, STAT3 is phosphorylated on S727, which regulates its mitochondrial localization (Wegrzyn J, et al. Science. 2009 323(5915):793-797). Cells expressing the Y705F mutant are viable and proliferate albeit at a slower rate compared to cells expressing WT-STAT3. Other studies have demonstrated that the STAT3-Y705F mutant can form dimers and the preformed unphosphorylated dimers were present in both stimulated and unstimulated cells (Mohr A, et al. Cell Commun Signal. 2013 11:83). Other STAT3 PTMs include acetylation on K685 by CBP/p300, S-glutathionylation by intracellular oxidative stress and trimethylation by EZH2 (Kim E, et al. Cancer Cell. 2013 23(6):839-852). STAT3 is constitutively phosphorylated on Y705 and S727 residues, acetylated on K685 and trimethylated on K180 (FIG. 9), but these modifications were interdependent in GBM cells. STAT3-KO completely abolished acetylation and trimethylation, and rescue of KO cells with WT-STAT3 restored these PTMs. While expression of the STAT3 mutants in KO cells did not significantly alter STAT3 acetylation at K685, expression of the Y705F mutant increased STAT3 trimethylation, but the S727A mutant decreased STAT3 trimethylation, again suggesting a central role of STAT3 Y705 phosphorylation in regulating STAT3 function. STAT3-S727 phosphorylation is dependent on STAT3-Y705 phosphorylation, but Y705 phosphorylation is independent of S727 phosphorylation (Ganguly D, et al. Oncotarget. 2018 9(31):22095-22112).

STAT3 has been shown to regulate autophagy through several mechanisms (You L, et al. Autophagy. 2015 11(5):729-739). Nuclear STAT3 regulates autophagy through the transcriptional regulation of pro-autophagy genes such as Beclin1 (BECN1) and anti- or pro-autophagy modulating microRNAs (You L, et al. Autophagy. 2015 11(5):729-739; Miao L J, et al. Tumour Biol. 2014 35(7):7097-7103). Our qPCR analyses of autophagy genes demonstrate that STAT3 inhibits ULK1 expression but increases expression of p62 and BNIP3 genes. STAT3-KO decreases BNIP3 and p62 protein levels but increases ULK1 expression in MT330 cells, showing a correlation between mRNA and protein levels. The BH3 domain-containing protein, BNIP3, is regulated by STAT3 phosphorylation. BNIP3 expression is linked with induction of autophagy and requires upregulation of concanavalin-induced JAK2/STAT3 signaling in GBM cells (Pratt J, et al. Cell Signal. 2014 26(5):917-924). In contrast, we found that STAT3-deletion and expression of the phosphorylation-inactive STAT3 mutants significantly decreased BNIP3 gene expression and protein levels showing that BNIP3 downregulation correlates with autophagy induction in GBM cells. These differences support the notion that BNIP3 plays diverse roles in GBM autophagy regulation, and these roles may be stimuli and cell context dependent.

Since enhanced autophagy results in p62 degradation and upregulation of Prom1 expression, we expected that induction of autophagy flux by STAT3 deletion would lead to enhanced degradation of p62 and increase Prom1 protein levels. Supporting this, STAT3-KO and Y705 mutant expressing cells showed reduced p62 protein levels by activation of autophagy flux. Control and WT-STAT3 expressing STAT3-KO cells had reduced autophagy flux and impaired autolysosomal p62 degradation in GBM cells. STAT3-KO and mutant expressing cells show upregulation of Prom1 protein levels, confirming autophagy induction. In addition, our results showed for the first time that p62 gene expression was significantly downregulated in these lines demonstrating that p62 is transcriptionally regulated by STAT3. Reconstitution of STAT3-KO cells with WT-STAT3 restored p62 gene expression confirming that STAT3 regulates p62 at the transcriptional level in MT330 cells. In contrast, p62 gene expression was unaltered in STAT3-KO and phosphorylation-defective mutant expressing LN229 lines. However, p62 protein levels were still reduced in these cells, suggesting transcription-independent but autophagy-dependent regulation of p62 in LN229 cells.

AMPKα maintains energy homeostasis and plays an important role in autophagy induction. AMPKα negatively regulates mTORC1 through TSC2, activates ULK1 Ser555 and Beclin1 Thr388 phosphorylation, all of which initiate autophagy (Zhang D, et al. Autophagy. 2016 12(9):1447-1459). ULK1 plays a central role in autophagy by promoting fusion of autophagosomes with lysosomes and phosphorylating multiple autophagy-related targets including Beclin1 and ATG101 (Wang C, et al. Nat Commun. 2018 9(1):3492). Data showed that the AMPKα/ULK1 signaling axis regulates STAT3-dependent autophagy in GBM cells. AMPKα and ULK1 phosphorylation is low in control EV GBM cells but was markedly increased in both STAT3-KO MT330 cells and cells expressing STAT3 phosphodeficient mutants. In LN229 and MT330 cells, STAT3-KO and Y705F mutant expressing cells showed elevated AMPKα activity. However, expression of the S727A mutant had no effect on AMPKα activity in LN229 cells but did so in MT330 cells, which suggests that Y705 phosphorylation regulates autophagy via AMPKα signaling in MT330 cells but not in LN229 cells. Rescue of KO cells with WT-STAT3 abolished AMPKα T172 phosphorylation confirming STAT3's role in repressing AMPKα signaling in both LN229 and MT330 GBM cell lines. Control EV MT330 cells were resistant to autophagy induction in response to mTOR inhibition (using Everolimus). These results demonstrate that the attenuating effect of STAT3 on autophagy induction primarily depends on Y705 phosphorylation and its ability to inhibit AMPKα signaling pathway. Furthermore, our results show STAT3 is a novel suppressor of ULK1 in MT330 cells. Knockout of STAT3 dramatically increases ULK1 protein and gene expression in MT330 cells, which were blocked by expression of WT-STAT3. This is consistent with earlier studies showing STAT1 as a transcriptional suppressor of autophagy through inhibition of ULK1 protein and mRNA levels (Goldberg A A, et al. J Biol Chem. 2017 292(5):1899-1909). In LN229 cells, STAT3-KO had no effect on ULK1 mRNA and protein levels but increased ULK1 activity, demonstrating that enhancement of ULK1 activity is transcription independent. These studies highlight the function of various phosphorylation-sites in STAT3 mutants, and our data unambiguously demonstrate that Y705F and S727A mutants differentially regulate AMPKα and ULK1 signaling to activate autophagy in GBM lines. In this context, our data also support the cellular and molecular heterogeneity seen between LN229 and MT330 GBM lines. This may account for the observed variability in AMPKα signaling in response to STAT3 deletion and expression of STAT3 mutants.

AMPKα activation inhibits mTORC1, which leads to autophagy. In addition to regulating mTORC1, AMPKα activation inhibits tumor cell growth by phosphorylating TSC2 on S1387, which in turn inhibits mTORC1 leading to autophagy activation (Inoki K, et al. Cell. 2003 115(5):577-590). These data demonstrate that cells lacking STAT3 and cells expressing STAT3 mutants have higher amounts of TSC2 T1462 and S1387 phosphorylation. The regulation of TSC2 and mTORC1 by AMPKα has special implications in autophagy regulation. While TSC2 T1462 phosphorylation inhibits its activity leading to mTORC1 activation (Di Nardo A, et al. Hum Mol Genet. 2014 23(14):3865-3874), AMPKα inhibits mTORC1 in part by phosphorylating and activating TSC2 on S1387.54 ULK1 S555 phosphorylation is mediated through AMPKα and indicates autophagy activation, whereas other ULK1 sites are targeted by mTOR to inhibit autophagy. 28 Conversely, ULK1 inhibits the kinase activity of mTORC1 to stimulate autophagy (Jung C H, et al. Autophagy. 2011 7(10):1212-1221). We find that AMPKα is activated in STAT3-KO and STAT3-KO GBM cells expressing phosphorylation-defective STAT3 mutants, which suggests that AMPKα directly phosphorylates ULK1 on several sites required to sustain ULK1 activation. This mechanism of ULK1 activation is sufficient to inhibit mTORC1 and activate autophagy. Future studies are needed to further define how STAT3 regulates AMPKα signalling, but our data suggest that activated AMPKα induces autophagy in STAT3-KO cells through TSC2 to directly activate ULK1.

Consistent with STAT3 suppressing autophagy through inhibition of AMPKα/ULKI signaling, inhibiting ULK1 activity by MRT68921 or ULK1 protein knockdown by siRNA decreased autophagy, and sensitized STAT3-KO and STAT3-phosphodeficient mutant expressing lines to caspase-3-dependent apoptosis. These results suggest that autophagy is cytoprotective in STAT3-KO cells. Thus, approaches directed at inhibiting ULK1 may inhibit autophagy through downstream blockade of ULK1-Atg14-Beclin1 signaling and consequently lead to GBM cell death. To our knowledge, this is the first example of the involvement of ULK1 signaling in the regulation of STAT3-dependent autophagy/apoptosis and highlight the targeting of both STAT3 and ULK1 as a potential novel therapeutic approach for GBM treatment.

### Example 2

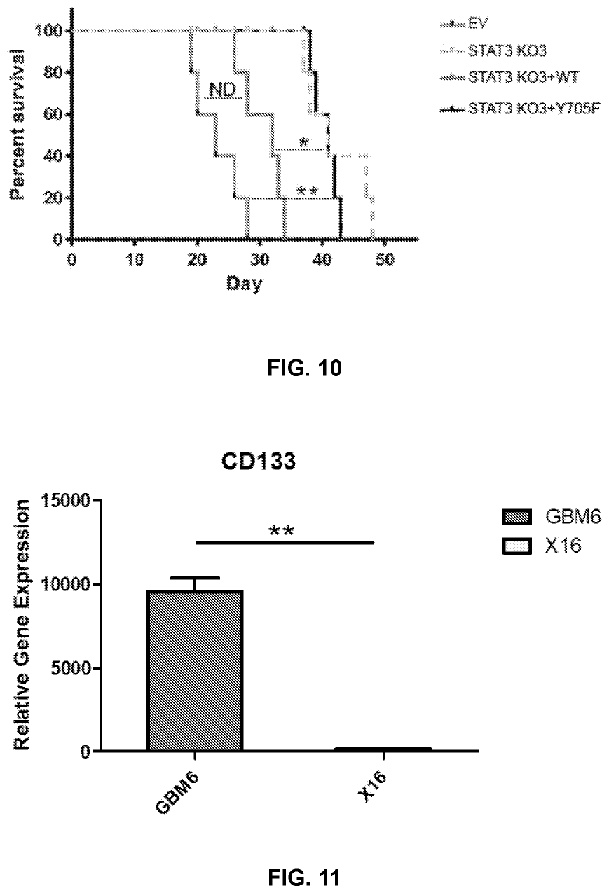

FIG. 10 shows Kaplan-Meier analysis of survival data in mice showing EV MT330 triggers GBM tumors and decreases survival in mice. STAT3-KO and Y705F-STAT3 mutant expressing MT330 cells increase survival in mice.

FIG. 11 shows CD133/Prom1 gene expression in GBM6 and X16 Glioblastoma Cancer Stem Cells.

FIG. 12 shows CD133/Prom1 cell surface expression in GBM6 and X16 Glioblastoma Cancer Stem Cells.

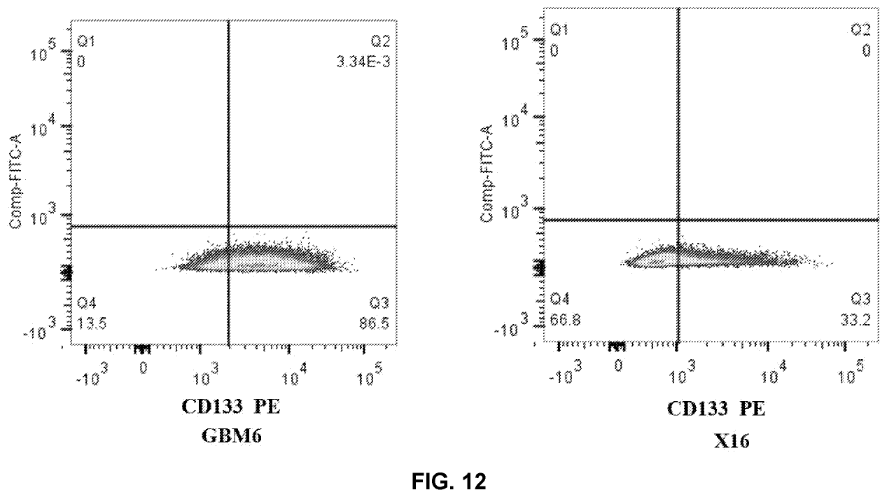

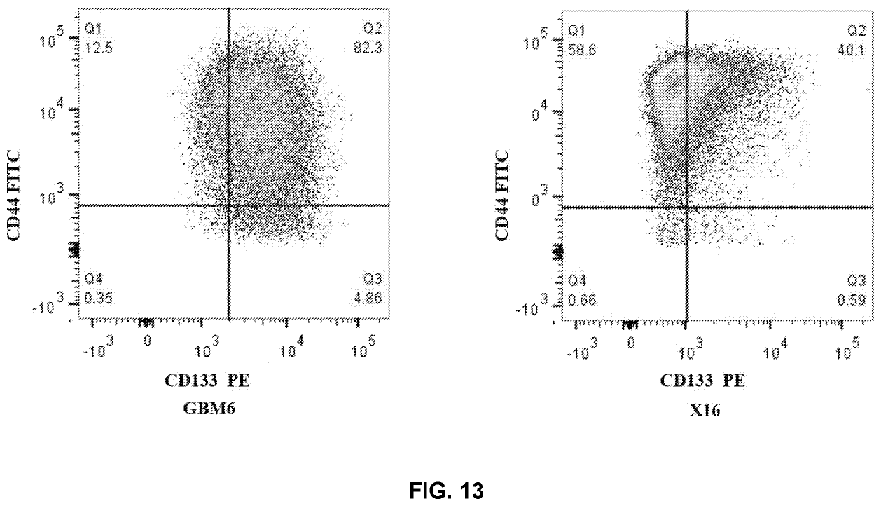

FIG. 13 shows CD133/Prom1 and CD44 cell surface expression in GBM6 and X16 Glioblastoma Cancer Stem Cells.

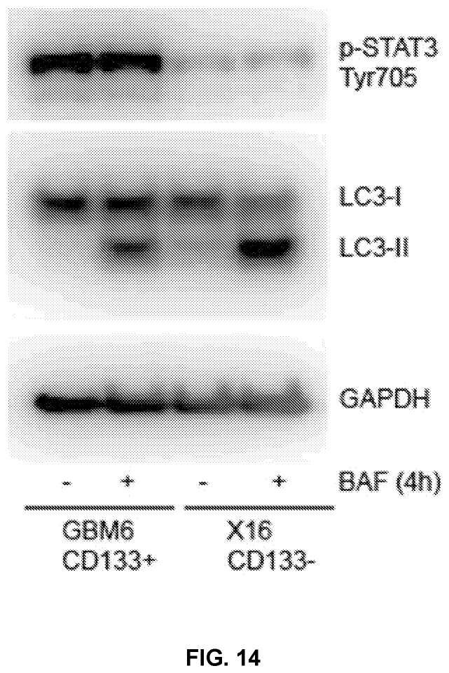

FIG. 14 shows CD133-ve glioblastoma stem cells (GSCs) have low pY705-STAT3, and elevated autophagy compared to CD133+ve GSCs suggesting that cell surface CD133 is negatively associated with autophagy in GSCs.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising a signal transducer and activator of transcription 3 (STAT3) inhibitor and one or more of a UNC51-like 1 (ULK1) inhibitor, histone deacetylase (HDAC) inhibitor, or an mTOR inhibitor in a pharmaceutically acceptable carrier.

2. The composition of claim 1, wherein the STAT3 inhibitor comprises WP1066.

3. The composition of claim 1, wherein the ULK1 inhibitor comprises MRT68921 or SB10206965.

4. The composition of claim 1, wherein the AMPKα inhibitor comprises Dorsomorphin.

5. The composition of claim 1, wherein the HDAC inhibitor comprises an HDAC 1, 2, 3, and 6 inhibitor.

6. The composition of claim 1, wherein the HDAC inhibitor comprises Vorinostat (SAHA).

7. The composition of claim 1, wherein the HDAC inhibitor comprises Trichostatin A.

8. The composition of claim 1, wherein the mTOR inhibitor comprises rapamycin or a rapalog.

9. The composition of claim 8, wherein the mTOR inhibitor comprises Everolimus.

10. The composition of claim 8, wherein the mTOR inhibitor comprises RapaLink.

11. A method for treating glioblastoma in a subject, comprising administering to the subject an effective amount of a signal transducer and activator of transcription 3 (STAT3) inhibitor and one or more of a ULK (UNC51-like) inhibitor, an AMPKα inhibitor, an histone deacetylase (HDAC) inhibitor, or an mTOR inhibitor.

12. The method of any claim 7 wherein the STAT3 inhibitor and the ULK1 inhibitor, AMPKα inhibitor, HDAC inhibitor, or mTOR inhibitor are administered concurrently.

13. The method of claim 7, wherein the STAT3 inhibitor and the ULK1 inhibitor, AMPKα inhibitor, HDAC inhibitor, or mTOR inhibitor are administered in the same composition.
